(19) United States
(12) Patent Application Publication
(10) Pub. No.: US 2026/0053422 A1
(43) Pub. Date: Feb. 26, 2026

(54) TRACKING REACTION TIME USING EAR-WORN DEVICES

(71) Applicant: ResMed Pty Ltd., Bella Vista (AU)

(72) Inventors: Liam Holley, Bella Vista (AU); Nicholas James MEALEY, Fort Lee, NJ (US)

(73) Assignee: ResMed Pty Ltd., Bella Vista (AU)

(21) Appl. No.: 19/304,754

(22) Filed: Aug. 20, 2025

Related U.S. Application Data
(60) Provisional application No. 63/685,325, filed on Aug. 21, 2024.
(60) Provisional application No. 63/685,324, filed on Aug. 21, 2024.
(60) Provisional application No. 63/685,322, filed on Aug. 21, 2024.
(60) Provisional application No. 63/813,243, filed on May 28, 2025.
(60) Provisional application No. 63/813,238, filed on May 28, 2025.
(60) Provisional application No. 63/813,222, filed on May 28, 2025.

(51) Int. Cl. A61B 5/375; A61B 5/00; A61B 5/11

(52) U.S. Cl. CPC A61B 5/375; A61B 5/1103; A61B 5/1118; A61B 5/681; A61B 5/6815; A61B 5/7282; A61B 5/746; A61B 2562/0219

(57) ABSTRACT

An ear-worn device may include an accelerometer and/or an electroencephalography (EEG) sensor to provide recommendations based on user activity and/or physiological responses. The ear-worn device may use the accelerometer to capture acceleration data to assess the wearer's movement rate, and detect changes. Using data from the EEG sensor, the processor may detect eyelid blinks and other signals of fatigue. The processor may generate a treatment recommendation based on any combination of the movement rate, changes, eyelid blinks, and/or signals of fatigue.

### CROSS-REFERENCE TO RELATED APPLICATIONS

This application claims the benefit of priority from U.S. Provisional Application No. 63/685,322, filed on Aug. 21, 2024, and from U.S. Provisional Application No. 63/685,324, filed on Aug. 21, 2024, and from U.S. Provisional Application No. 63/685,325, filed on Aug. 21, 2024, and from U.S. Provisional Application No. 63/813,222, filed on May 28, 2025, and from U.S. Provisional Application No. 63/813,238, filed on May 28, 2025, and from U.S. Provisional Application No. 63/813,243, filed on May 28, 2025, the entirety of each of which is hereby incorporated by reference.

### BACKGROUND

Wearable devices have become increasingly popular in health and fitness applications. However, accurately utilizing data from wearables to derive meaningful health-related insights presents several challenges. One issue is tracking changes in reaction time, as changing reaction times may be indicative of various pathologies. Furthermore, changing reaction time may be associated with fatigue, which may pose dangers in various contexts. Therefore, there is a need to track reaction time using wearable devices.

### SUMMARY

In various embodiments, a system comprises a first ear-worn device and a second ear-worn device. The first ear-worn device includes an accelerometer that provides acceleration data, alongside a processor that executes specific instructions. These instructions enable the processor to determine the duration of a body movement wearing the first ear-worn device based on the acceleration data. From this duration and a profile, the processor identifies a change in the duration of the movement. Subsequently, the processor generates a recommendation based on the change in duration and outputs an indication of this recommendation.

In some embodiments, a non-transitory computer-readable storage medium contains instructions that, when executed by a processor of an ear-worn device, perform a sequence of operations. The processor receives acceleration data from the device's accelerometer, determines the duration of a user's movement, and then identifies any changes in this duration using a profile. Based on these changes, the processor generates a recommendation and outputs an indication of this recommendation.

In some embodiments, a method involves specific steps carried out by a processor within an ear-worn device. The processor receives acceleration data from its accelerometer, determines the movement duration of a body wearing the device, and then assesses changes in this duration using a profile. Based on the identified changes, the processor generates a recommendation and outputs an indication of this recommendation.

The methods, systems, devices, and apparatuses described may be implemented to improve the functionality of a processor, such as a processor of a specific purpose computer, wearable device, respiratory monitor, and/or a respiratory therapy apparatus. Moreover, the described methods, systems, devices, and apparatus can provide improvements in the technological field of automated detection, management, monitoring, and/or treatment of health conditions, including, for example, sleep disordered breathing, fatigue, or other conditions where declining capabilities are a symptom.

### BRIEF DESCRIPTION OF THE SEVERAL VIEWS OF THE DRAWINGS

FIG. 1 illustrates a system that uses earbuds to track reaction time, in accordance with one embodiment.

FIG. 2 illustrates components of an earbud that tracks reaction time, in accordance with one embodiment.

FIG. 3 illustrates an aspect of the subject matter in accordance with one embodiment.

FIG. 4 illustrates an aspect of the subject matter in accordance with one or more embodiments.

FIG. 5A-FIG. 5B show a patient tracking device worn by a patient.

FIG. 6 shows a patient tracking device worn by a patient together with a patient interface.

FIG. 7 illustrates an aspect of the subject matter in accordance with one embodiment.

FIG. 8 illustrates an aspect of the subject matter in accordance with one embodiment.

FIG. 9 illustrates a logic flow 900 in accordance with one embodiment.

FIG. 10 illustrates a logic flow 1000 in accordance with one embodiment.

FIG. 11 illustrates a logic flow 1100 in accordance with one embodiment.

FIG. 12 illustrates a logic flow 1200 in accordance with one embodiment.

FIG. 13 illustrates a logic flow 1300 in accordance with one embodiment.

FIG. 14 illustrates a logic flow 1400 in accordance with one embodiment.

FIG. 15 illustrates a logic flow 1500 in accordance with one embodiment.

FIG. 16 shows a patient interface in the form of a nasal mask.

FIG. 17A-FIG. 17D illustrate components of a Respiratory Pressure Therapy (RPT) device.

FIG. 18 illustrates a computing system 1800 in accordance with one embodiment.

### DETAILED DESCRIPTION

Embodiments disclosed herein include techniques for using ear-worn devices to track the reaction time of a person. The reaction time of a person may be based on any suitable factor and/or combination of factors, such as how often the eyes blink, brain activity, body stability, movements, sleep patterns, falls, and the like. For example, an ear-worn device, such as one or more earbuds, may include an accelerometer and an electroencephalography (EEG) sensor. A processor of the earbud may use data from the accelerometer and/or the EEG sensor to monitor a patient over time and generate recommendations based on the monitoring.

For example, the EEG sensors may be used to monitor eye blinks over time, as an eye blink may appear in the data collected by the EEG sensors. Similarly, the EEG sensors may be used to monitor electrical activity of the brain. Some features, e.g., increased alpha waves from the brain, may be associated with drowsiness or fatigue. Therefore, based on the monitoring of eye blinks and/or the electrical activity of the brain, the processor of the earbud may determine the person is experiencing a slower reaction time, e.g., is fatigued, drowsy, falling asleep, etc.

In addition and/or alternatively, the accelerometers may be used to track the position of the head and/or any movements of the head over time. For example, if the accelerometers indicate the head is periodically tilting forward and down, the processor may determine the person is falling asleep.

In some embodiments, accelerometers of other devices may be used to provide position and/or movement information for other parts of the body. For example, a driver who is tired and/or falling asleep may have slower reaction time, which causes the driver to make sudden, rapid movements of the steering wheel. These movements may be captured by wearables such as a smartwatch. The data from the smartwatch may be transmitted to the processor of the earbud, which may determine the person is falling asleep based further on the data from the smartwatch.

Embodiments disclosed herein may generate any number and type of recommendations based on monitoring a person. For example, based on a determination that a person is drowsy, fatigued, falling asleep, etc., while driving, the earbuds may determine to generate a notification to alert the driver and instruct the driver to stop driving. As another example, the earbuds may transmit an instruction to a computing device of the vehicle to engage autonomous driving features (or any other type of driver assistance features, such as lane assist, adaptive cruise control, etc.).

In some embodiments, the earbuds may be used to detect pathologies. For example, a person who has a disease such as dementia may restrict the ability of the body to make certain types of movements. For example, a person with Lewy body dementia may generally be more rigid, which causes the person to make slower movements, make fewer movements, etc. For example, a person with dementia may require more time to get out of bed, sit down, stand up, etc. By tracking a person's movements over time, a change in reaction time may be detected. By detecting the change in reaction time, the earbuds may determine that the person is afflicted by a pathology, e.g., dementia. The earbuds may output an indication of the pathology, e.g., by transmitting a data package to the person's medical provider which indicates, based on the collected data and determined changes, the person may have the pathology.

In some embodiments, the earbuds may detect pathologies based at least in part on environmental conditions. For example, a person who has Alzheimer's may be more susceptible to fatigue in certain environmental conditions, e.g., in rooms with multiple people speaking, loud rooms, etc. However, people without the condition may not experience these levels of fatigue. Therefore, by monitoring the environment and the person, the earbuds may detect a pathology (e.g., Alzheimer's) of the person.

The earbuds may further be used to predict certain events, such as falls, accidents, etc. For example, by monitoring the movements of a person over time, the earbuds may determine that the person is exhibiting poor balance and may have a pathology. Based on the determination that the person has poor balance and may have a pathology, the earbuds may generate a recommendation. For example, the recommendation may be an audible alert instructing the person to sit down, use a cane or walker, etc. In some embodiments, the earbuds may transmit a recommendation to a medical provider's system to prescribe an assistive device such as a walker, cane, wheelchair, etc.

In some embodiments, based on monitoring a person over time, the earbuds may generate recommendations to modify respiratory therapy systems used by the person. For example, the earbuds may recommend changing the type of mask used as part of a respiratory therapy system, change operating parameters of the respiratory therapy system (e.g., modifying the therapy pressure provided by Continuous Positive Airway Pressure (CPAP) device), changing the type of respiratory therapy system, etc. In some embodiments, the earbuds may programmatically initiate the recommendations, e.g., by transmitting an instruction to a respiratory therapy device to modify one or more operational parameters (e.g., adjusting titration, pressure, etc.), transmitting an indication to a medical provider to prescribe a respiratory therapy device, placing an order for new and/or replacement parts, etc.

Advantageously, embodiments disclosed herein provide techniques to identify, in real time, factors that may impact the health of a person and affect a treatment or prophylaxis thereof. By leveraging sensors integrated into wearable devices that can identify the movements, fatigue levels, and/or reaction time of patients, a precise determination of adverse health events may be generated. When the earbuds detect an adverse health event, the earbuds may recommend a treatment to improve the health of the patient. For example, embodiments disclosed herein may determine a particular recommendation, e.g., waking the person, recommending therapies and/or devices, modifying parameters for respiratory therapy systems, reordering supplies, and affecting the recommendation to improve the health of the patient. Because some conditions may have severe (or even fatal) health consequences, real-time detection may provide opportunities to avoid these outcomes. Embodiments are not limited in these contexts.

Aspects of the present disclosure and certain features, advantages, and details thereof are explained more fully below with reference to the non-limiting examples illustrated in the accompanying drawings. Descriptions of well-known processing techniques, systems, components, etc. are omitted to not unnecessarily obscure the disclosure in detail. The detailed description and the specific examples, while indicating aspects of the disclosure, are given by way of illustration only, and not by way of limitation. Various substitutions, modifications, additions, and/or arrangements, within the spirit and/or scope of the disclosed aspects will be apparent to those skilled in the art from this disclosure. Note further that numerous aspects and features are disclosed herein, and unless inconsistent, each disclosed aspect or feature is combinable with any other disclosed aspect or feature as desired for a particular embodiment of the concepts disclosed herein.

Unless described or implied as exclusive alternatives, features throughout the drawings and descriptions should be taken as cumulative, such that features expressly associated with some particular embodiments can be combined with other embodiments. Like numbers refer to like elements throughout.

While certain exemplary embodiments have been described and shown in the accompanying drawings, it is to be understood that such embodiments are merely illustrative of, and not restrictive on, the broad disclosure, and that this disclosure not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the herein described embodiments can be configured without departing from the scope and spirit of the disclosure. Therefore, it is to be understood that, within the scope of the included claims, the disclosure may be practiced other than as specifically described herein.

Additionally, illustrative embodiments are described below using specific code, designs, architectures, protocols, layouts, schematics, or tools only as examples, and not by way of limitation. Furthermore, the illustrative embodiments are described in certain instances using particular software, tools, or data processing environments only as example for clarity of description. The illustrative embodiments can be used in conjunction with other comparable or similarly purposed structures, systems, applications, or architectures. One or more aspects of an illustrative embodiment can be implemented in hardware, software, or a combination thereof.

As understood by one skilled in the art, program code, as referred to in this application, can include both software and hardware. For example, program code in certain embodiments of the present disclosure can include fixed function hardware, while other embodiments can utilize a software-based implementation of the functionality described. Certain embodiments combine both types of program code.

The terms “coupled,” “fixed,” “attached to,” “communicatively coupled to,” “operatively coupled to,” and the like refer to both (i) direct connecting, coupling, fixing, attaching, communicatively coupling; and (ii) indirect connecting coupling, fixing, attaching, communicatively coupling via one or more intermediate components or features, unless otherwise specified herein. “Communicatively coupled to” and “operatively coupled to” can refer to physically and/or electrically related components.

Some of the figures may include a logic flow. Although such figures presented herein may include a particular logic flow, it can be appreciated that the logic flow merely provides an example of how the general functionality as described herein can be implemented. Further, a given logic flow does not necessarily have to be executed in the order presented unless otherwise indicated. Moreover, not all acts illustrated in a logic flow may be required in some embodiments. In addition, the given logic flow may be implemented by a hardware element, a software element executed by a processor, or any combination thereof. The embodiments are not limited in this context.

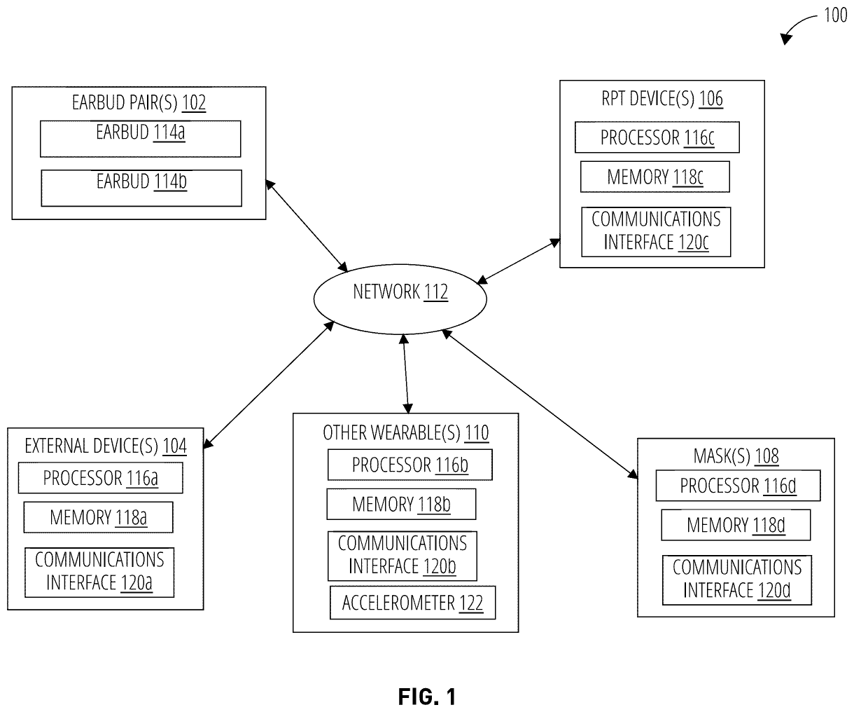

FIG. 1 illustrates a system 100 in accordance with one embodiment. The system 100 may be a system that uses earbuds to monitor patients. By monitoring the patients, the earbuds may track the reaction time of patients. Therefore, one or more components of the system 100 may be part of a patient health system. Embodiments are not limited in these contexts.

As shown, the system 100 includes one or more earbud pairs 102, one or more external devices 104, one or more respiratory pressure therapy (RPT) devices 106, one or more masks 108, and one or more other wearables 110 communicably coupled via a communications network 112.

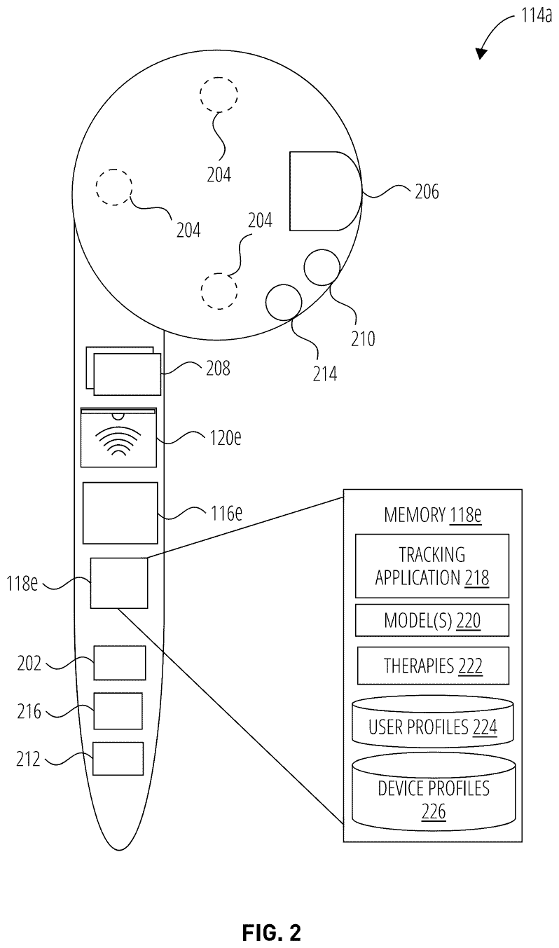

The earbud pair 102 includes an earbud 114a and an earbud 114b. Additional components of the earbuds 114a-114b are depicted in FIG. 2. Generally, earbuds 114a-114b are worn in, around, or proximate to the ear of a person. Although the “earbud” is used as one reference example herein, the disclosure is equally applicable to other types of ear-worn electronic devices. Therefore, embodiments are not limited to the earbud form factor.

The external devices 104 represent any type of device, such as a computing device, smartphone, laptop, tablet, hub, smart home device, medical provider device or system, medical device, networking device, Internet of things (IoT) device, vehicle computing systems, and the like. The other wearables 110 represent any type of wearable device, such as smartwatches, devices worn on the torso (e.g., heartrate monitors), smart rings, smart goggles, smart glasses, step counters, medical devices, straps, ankle or leg-worn devices, and the like.

The RPT device 106 represents any respiratory therapy device or system, such as a Continuous Positive Airway Pressure (CPAP) device. More generally, the RPT device 106 is configured to generate a flow of air for delivery to the human airways via an interface such as a mask 108. In some embodiments, the RPT devices 106 include RPT devices that are implanted at least partially within the body of a patient.

As shown in FIG. 1, the external devices 104, RPT devices 106, masks 108, and other wearables 110 include a processor 116a, a processor 116b, a processor 116c, and a processor 116d, respectively. As shown in FIG. 2, the earbud 114a, which represents earbud 114b, similarly includes a processor 116e, a memory 118e, and a communications interface 120c. Furthermore, the other wearables 110 include an accelerometer 122, while the earbuds 114a-114b each include a respective accelerometer 216. An accelerometer such as accelerometer 122 or accelerometer 216 may measure the rate of change of velocity (e.g., acceleration) of the device (e.g., the other wearables 110 and earbuds 114a, 114b, respectively) along one or more axes, providing data on movement and orientation.

The processors 116a-116e represent any type of processor circuit. Examples of processor circuits include an Intel® x86 processor, an ARM® processor, a RISC processor, AMD® processors, and similar processors. Similarly, the external devices 104, RPT devices 106, masks 108, and other wearables 110 include a memory 118a, a memory 118b, a memory 118c, and a memory 118d, respectively. The memories 118a-118e represent any type of computer memory, such as volatile memory or non-volatile memory. To communicate via the network 112, the external devices 104, RPT devices 106, masks 108, and other wearables 110 include a communications interface 120a, a communications interface 120b, a communications interface 120c, and a communications interface 120d, respectively. The communications interfaces 120a-120e represent any type of data communications interface, such as a wireless (or wired) transceiver.

The network 112 may be any type of data communications network. In some embodiments, the network 112 is a wireless communications network. Examples of wireless communications networks include an IEEE 802.11 wireless network, Wi-Fi, Bluetooth®, Bluetooth Low Energy (BLE), near-field communication (NFC), radio frequency identification (RFID), radio frequency (RF) networks, or any other type of wireless communication network. Therefore, the communications interfaces 120a-120e are configured to support IEEE 802.11 wireless networks, Wi-Fi, Bluetooth, Bluetooth Low Energy (BLE), near-field communication (NFC), radio frequency identification (RFID), radio frequency (RF) networks, or any other type of wireless communication network. Furthermore, the network 112 represents direct wireless communications between the entities of the system 100.

Collectively, the components of the system 100 (or any subset thereof) are configured to monitor a human patient, collect data from the patient, determine and record reaction times of the patient, detect pathologies, predict adverse health events, modify therapies delivered to the patient, and/or generate recommendations.

For example, the earbuds 114a-114b may collect data from the patient over time, determine the reaction time of the patient is slowing (e.g., because the patient is falling asleep), and generate one or more recommendations. In some embodiments, the collected data may include data from the external devices 104, other wearables 110, masks 108, and/or RPT devices 106. In some embodiments, the recommendation is outputted by the earbuds 114a-114b to the patient, e.g., as an audible alert, vibrations, etc. Doing so may wake the patient. For example, by determining a person driving a car is falling asleep, the earbuds may wake the person, engage autonomous driving features of the car, etc., which may avoid an accident and associated adverse health events.

As another example, based on the data collected by the earbuds 114a-114b, the earbuds 114a-114b may determine that certain capabilities of the patient are decreasing. For example, the earbuds 114a-114b may determine the patient takes longer to complete certain movements (or tasks), has poorer balance (e.g., falls more frequently), has trouble has slower reaction times, gets fatigued more easily (or in environments where others do not experience fatigue), etc. As such, the earbuds 114a-114b may determine the patient has a pathology, and generate a recommendation. For example, the earbuds 114a-114b may output an audible message indicating the detected issues, cause a notification to appear on the user's smartphone indicating the detected issues, transmit a message to the patient's medical provider, etc.

In some embodiments, the recommendation is transmitted by earbud 114a or 114b via the network 112. For example, the recommendation may include prescribing a device to assist with walking (e.g., cane, walker, wheelchair, etc.), prescribing different type of mask 108, prescribing a different type of RPT device 106, modifying parameters of the RPT device 106 (e.g., changing pressure, titration, etc.), etc. In some embodiments, the recommendation may be sent to an external device 104, such as a medical provider system. Doing so allows the medical provider to prescribe a therapy for the patient, e.g., to prescribe a walking cane, prescribe a different type of mask 108, change RPT devices 106 (e.g., CPAP, bilevel, etc.), change attributes of the therapy provided by the RPT device 106, etc. In another example, the alert may be sent as an instruction to the RPT device 106 and/or the mask 108. For example, the RPT device 106 may modify, based on the instruction, the type of therapy, attributes of the therapy (e.g., increase pressure, decrease pressure, modify titration, etc.), and/or a duration of the therapy provided to the patient. As stated, in some embodiments, the external devices 104 may include vehicle computing systems. Therefore, the earbuds 114a, 114b may transmit an indication to the vehicle computing system, e.g., to engage safety features such as autonomous driving, semi-autonomous driving, lane assist, adaptive cruise control, etc. Embodiments are not limited in these contexts.

FIG. 2 illustrates an example earbud 114a that can communicatively couple with earbud 114b to form a pair of untethered, wireless earbuds according to some embodiments of the present technology. Although earbud 114a is depicted, earbud 114b includes the components depicted in FIG. 2.

The earbud 114a uses communications interface 120e to communicatively couple with another wireless earbud, e.g., earbud 114b, and to pair with a source device, e.g., a companion communication device (e.g., external devices 104 such as smartphones, other wearables 110, etc.) that can provide audio data that the earbuds 114a can reproduce as audio signals for a user of the earbuds 114a, 114b. In some embodiments, a process of pairing the earbuds 114a, 114b is initiated when the earbuds 114a, 114b are contained within a housing/case, not pictured for clarity. In some circumstances, once a pairing mode is enabled for the earbuds 114a, 114b, the earbuds 114a, 114b remain in the enabled pairing mode until one or more of the following occurs: (i) the earbud 114a or 114b pairs with a companion communication device, (ii) a pairing mode of the earbuds 114a, 114b times out (e.g., the earbud 114a or 114b does not pair with a companion communication device within a fixed time period, such as thirty seconds), (iii) the earbud 114a or 114b is removed from the case, (iv) the wireless earbud case commands one or more both of the earbuds 114a, 114b to exit the pairing mode, or (v) the companion communication device commands the earbuds 114a, 114b to exit the pairing mode. The earbud 114a can also include a battery 202 and sensors 204 for detecting a wearing status of the earbud 114a, e.g., when the earbud 114a is placed in and/or removed from an ear, whether the earbud 114a is in a user's ear, e.g., an in-ear wearing status, or is not in a user's ear, e.g., an out-of-ear wearing status.

Additionally, the earbud 114a includes one or more audio output devices such as a speaker 206 for converting a received signal, e.g., which can include audio data, into audible sound. The signal can be received from a paired companion communication device via the communications interface 120e. The memory 118e in the earbud 114a stores firmware for operating the earbud 114a as well as data for coupling with other wireless earbuds and for pairing the earbud 114a with companion communication devices. For example, the memory 118e in the earbud 114a can store a connection history for companion communication devices with which the earbud 114a has previously paired. The connection history can include data for automatically pairing the earbud 114a with the companion communication device without having to configure a connection between the earbud 114a and the companion communication device (e.g., enter a password, exchange shared secrets, etc.). For example, the connection history can include one or more link keys for connecting to a wireless network such as network 112 (e.g., Bluetooth link keys). The memory 118e of the earbud 114a can also store a MAC address that uniquely identifies the earbud 114a as well as store a paired partner MAC address of another wireless earbud 114b that has previously coupled with the earbud 114a. The memory 118e also stores instructions that, when executed by the processor, causes the earbud 114a to communicatively couple with another wireless earbud.

As shown, the earbud 114a includes one or more sensors 204, one or more speakers 206, two or more microphone arrays 208, a haptic feedback module 212, an accelerometer 216, a pulse oximeter 210, and one or more electrodes 214. The speakers 206 are devices to output audio, e.g., soundwaves. Each of the microphone arrays 208 includes a plurality of microphones (not pictured) that are configured to detect and record audio data, e.g., soundwaves. Therefore, a given earbud 114a, 114b, may include a plurality of microphone arrays 208, with each microphone array 208 including a plurality of microphones. The total number of microphones in each earbud 114a, earbud 114b may, therefore, number in the tens, hundreds, thousands, or more. In some embodiments, a first one of the microphone arrays 208 is located at a first end of the earbud 114a (e.g., nearest to the ear canal), while a second one of the microphone arrays 208 is located at an opposite end of the earbud 114a (e.g., farthest from the ear canal). In such embodiments, one or more other microphone arrays 208 may be located between the first and second microphone arrays 208. Embodiments are not limited in these contexts.

The haptic feedback module 212 is a device that generates vibrations or other tactile sensations, such as piezoelectric actuators/sensors, etc. The haptic feedback module 212 may detect reflections thereof, e.g., reflections of vibrations from the ear canal when the earbud 114a is worn by a patient, which may be useful in tracking reaction times of the patient, detecting a pathology in the patient, and/or detecting a therapy device used by the patient. The haptic feedback module 212 may further output vibrations or other haptic feedback to cause a patient to wake up, change sleep positions, etc.

The sensors 204 represent any type of sensor, such as a pressure sensor, a flow rate sensor, a temperature sensor, a motion sensor, a camera, an infrared (IR) sensor, a photoplethysmogram (PPG) sensor, an electrocardiogram (ECG) sensor, a capacitive sensor, an electromyography (EMG) sensor, an oxygen sensor, an analyte sensor, a gyroscope, a magnetometer, a moisture sensor, a light detection and ranging (LiDAR) sensor, a galvanic skin response (GSR) sensor, or a carbon dioxide (CO2) sensor. The pulse oximeter 210 is a peripheral oxygen saturation sensor that is configured to determine a peripheral oxygen saturation (SpO₂) value of a bloodstream of a patient. Stated differently, the pulse oximeter 210 is configured to detect the oxygen levels of a patient.

The one or more electrodes 214 may be any type of electrode to detect electrical signals by contacting the skin of a person. For example, the electrodes 214 may be electroencephalography (EEG) electrodes (also referred to as “EEG sensors”) and/or electrooculography (EOG) electrodes (also referred to as “EOG sensors”). For example, by contacting the skin of the ear, the electrodes 214 may detect the electrical activity of the brain (e.g., the electrical signals generated by the neurons of the brain). Similarly, eye blinks may create electrical signals that are detected by the electrodes 214. In some embodiments, the eye blinks and brain activity may be present in the same signals captured by the electrodes 214. Therefore, in such embodiments, the processor 116e may filter out the eye blinks and/or brain activity from the signals captured by the electrodes 214, e.g., using filtering algorithms, etc.

As shown, the memory 118e of the earbud 114a includes a tracking application 218, one or more models 220, a data store of therapies 222, a data store of user profiles 224, and a data store of device profiles 226. The tracking application 218 is generally configured to monitor a patient over time. The monitoring may include, but is not limited to, monitoring any attribute of a patient such as eye blinks, movements, body positions (and/or positions of body parts), sleep patterns, balance, gait tracking, falls, etc. Furthermore, based on the monitoring, the tracking application 218 may determine a reaction (or movement) time of the person, detect changes in the reaction or movement time of the person, detect fatigue (or different levels of fatigue), etc.

For example, as the tracking application 218 tracks the movements, actions, etc., of a person, the tracking application 218 may determine a type of movement, a duration of the movement (e.g., based at least in part on a start timestamp and an end timestamp), and any other attributes associated with the movement (e.g., if a fall is detected, physiological attributes of the person during the movement, etc.) and store indications of the same in the user profile 224 for the patient. Over time, the tracking application 218 may determine changes in the movements, e.g., based on the user profile 224. For example, based on one or more entries in the user profile 224, the tracking application 218 may determine that a patient requires 5 seconds to complete a task (e.g., an average time of all entries in the user profile 224 and/or based on specific entries in the user profile 224) such as getting out of bed. The tracking application 218 may then detect the task, e.g., based on the accelerometers 216, time of day, sleep patterns, classifications, etc., and determine the patient required 10 seconds to get out of bed. The tracking application 218 may then determine the change in the amount of time, and generate a recommendation. For example, the tracking application 218 may diagnose a pathology, output an indication of the change in reaction time to the patient, transmit an indication of the change and/or pathology to the patient's medical provider, output an instruction to the patient to use an assistive walking device, etc. In some embodiments, the tracking application 218 may continue to monitor the patient to detect and analyze the movements. For example, the tracking application 218 may monitor the amount of time the patient requires to get out of bed for a predetermined number of days, weeks, months, etc., prior to diagnosing a pathology, generating a recommendation, etc. Regardless of the time interval used by the tracking application 218, by detecting movement durations, and comparing these durations to stored durations in the user profile 224, the tracking application 218 may detect changes.

The tracking application 218 may generally track movements and/or positions of a person based at least in part on the data from the accelerometer 216 of the earbud 114a and/or the accelerometer 216 of earbud 114b. In addition and/or alternatively, the tracking application 218 may use data captured by the electrodes 214 to detect brain activity, eye blinks, or other electrical artifacts indicative of the reaction time of the person. For example, by analyzing electrical activity of the brain via signals collected by the electrodes 214, the tracking application 218 may detect alterations in the power and/or distribution of certain frequency bands associated with brain activity, such as alpha waves (approximately 8-13 Hertz (Hz)), theta waves (approximately 4-7 Hz), and beta waves (approximately 13-30 Hz). The tracking application 218 may record indications of the monitoring and/or any attribute thereof (including associated timestamps and/or detected movements) in the user profiles 224 periodically over time.

Generally, alpha waves may be most prominent during relaxed wakefulness with closed eyes. Theta waves may be associated with drowsiness and early stages of sleep. Theta waves may increase with mental or physical fatigue. Beta waves may be associated with active thinking, problem-solving, and focused attention. Fatigue may lead to an increase in alpha and/or theta wave power in the frontal and central brain regions, indicating compensatory mechanisms and reduced cognitive alertness. Fatigue may also cause a decrease in beta wave power, reflecting diminished cognitive engagement. As fatigue intensifies, alpha and beta may rhythms slow, signifying reduced neural activity. Under fatigue, EEG signals may become more variable, demonstrating unstable arousal states with alternating theta, low-frequency alpha, and beta activity. As fatigue intensifies, the frequency of alpha and beta waves may shift toward the lower end of their respective bands. Fatigue may impact event-related potentials, resulting in delayed and reduced event-related potential (ERP) component amplitudes, highlighting slower and less effective cognitive processing. For example, fatigue may negatively affect ERPs, which are the brain's response to specific stimuli, e.g., visual and/or auditory stimuli.

Therefore, by monitoring brain activity captured by the electrodes 214 according to these factors, the tracking application 218 may detect fatigue and/or the onset of sleep. Furthermore, by tracking these factors in a person over time, the tracking application 218 may detect the onset of fatigue and/or sleep. For example, if the tracking application 218 detects one or more of: (i) an increase in alpha wave power, (ii) an increase in theta wave power, (iii) a decrease in beta wave power, (iv) a decrease in the frequencies of alpha waves, (v) a decrease in the frequencies of beta waves, (vi) periodic bursts of high theta or low-frequency alpha alternating with brief moments of increased beta activity, or (vii) delayed and reduced amplitude of ERP components, the tracking application 218 may determine the person is experiencing slower reaction times, is fatigued and/or is falling asleep. In some embodiments, the tracking application 218 may compute a fatigue score based on one or more of these factors. For example, the tracking application 218 may compute a score based on an algorithm that weights each factor and produces a score in a predetermined range (e.g., 0-100). The scores may be categorized into different fatigue levels (e.g., low from 80-100, medium from 60-79, high under 60, etc.).

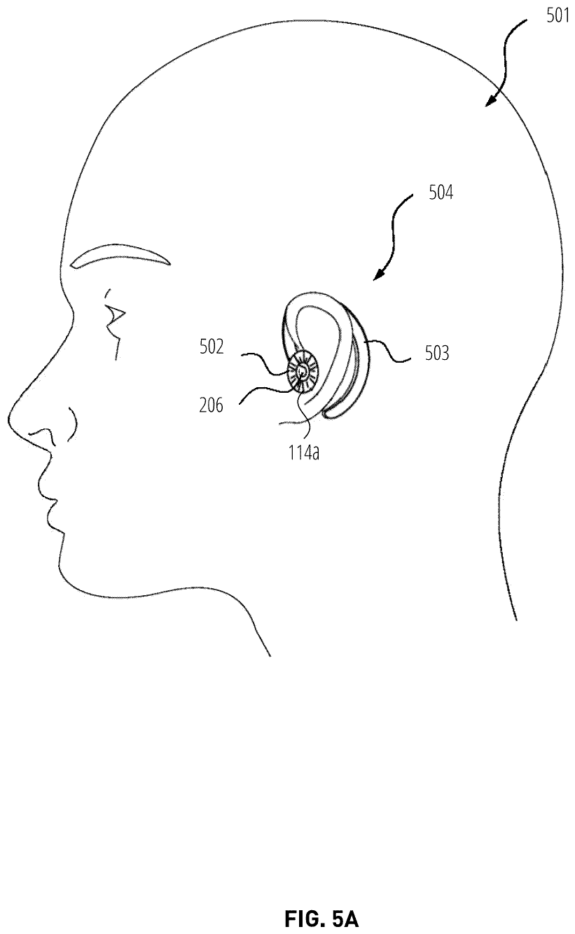

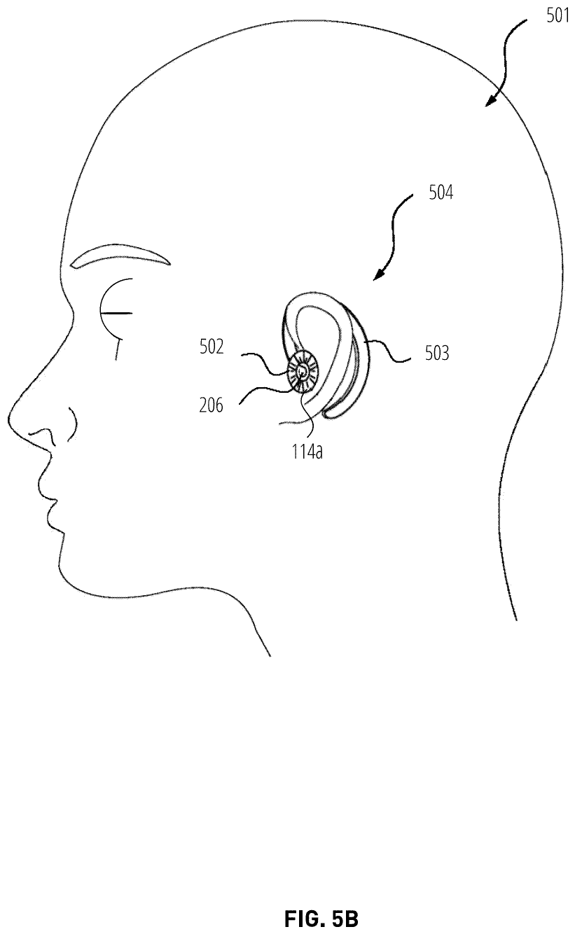

As stated, the tracking application 218 may further use the electrodes 214 to detect eye blinks and record indications of eye blinks in the user profile 224 over time. For example, as shown in FIG. 5A, the eye blinks of a patient 501 wearing earbud 114a may be monitored. Because the patient 501 depicted in FIG. 5A is not blinking, the electrodes 214 may only detect brain activity in EEG signals. However, when the patient 501 blinks, as depicted in FIG. 5B, the blink creates a transient potential difference because the eye acts like a dipole, with the cornea being positively charged relative to the retina. Stated differently, when the eyelid moves during a blink, it alters the electrical field, producing a measurable change in voltage in the EEG signals detected by the electrodes 214. In some embodiments, the eye blink is detected as an artifact in the brain signals. In such embodiments, the tracking application 218 may detect the eye blinks based on the artifacts.

In some embodiments, the user profiles 224 include threshold eye blinks of a person while awake. For example, by monitoring eye blinks and storing indications of eye blinks in the user profiles 224, the tracking application 218 may determine the person blinks 18 times per minute while awake. Therefore, if the tracking application 218 determines the person is blinking 10 times per minute, the tracking application 218 may determine the person is fatigued and generate a recommendation. For example, the recommendation may include outputting an audible alert via the speakers 206 indicating the fatigue and to recommend resting. As another example, the tracking application 218 may transmit a notification to the person's smartphone (and/or other wearables 110) indicating the fatigue and recommending resting.

As another example, the tracking application 218 may monitor the length of individual eye blinks (e.g., in milliseconds, etc.) and record indications of the length of each eye blink in the user profile 224 over time. In some embodiments, the user profiles 224 include threshold eye blink lengths of a person while awake. For example, by monitoring eye blinks and storing indications of eye blink lengths in the user profiles 224, the tracking application 218 may determine the person's average eye blink lasts 100 milliseconds. Therefore, if the tracking application 218 determines the blinks last 200 milliseconds, the tracking application 218 may determine the person is fatigued and generate a recommendation.

In some embodiments, the tracking application 218 may consider movements when generating a recommendation, detecting fatigue, and/or detecting a pathology. For example, if the accelerometers 216 indicate a rate of movement associated with driving a car (e.g., 60 kilometers per hour), the tracking application 218 may determine to generate a recommendation to alert the person of the fatigue and stop driving. Similarly, the tracking application 218 may transmit an instruction to the vehicle (e.g., one of the external devices 104) to engage safety features such as autonomous driving, etc.

More generally, data from the accelerometers 216 of the earbuds 114a-114b and/or the accelerometers 122 of the other wearables 110 may be used by the tracking application 218, e.g., to detect movement patterns, contributing to activity tracking and enhancing user safety in dynamic environments. In some embodiments, the accelerometer 216 includes other features such as a gyroscope to measure angular velocity (e.g., rotation) to detect changes in orientation. In some embodiments, the data from the accelerometers 216 and/or 122 is used to determine the position of the person's body and/or body parts. In some embodiments, the user profiles 224 include features associated with data from accelerometers that are associated with different orientations and/or movements. For example, the data from the accelerometers 216 may indicate the person's head is upright and facing forward. However, over time, the data from the accelerometers 216 may indicate the person's head is nodding forward as they doze off to sleep. Therefore, the tracking application 218 may determine the person is fatigued and/or falling asleep.

More generally, any activity may be detected using data from the accelerometers 122 and/or accelerometers 216. For example, if the data from the accelerometers 122 and/or accelerometers 216 indicate the person is lying on their back, the tracking application 218 may determine the person is sleeping. As another example, the data from the accelerometers 122 and/or accelerometers 216 may indicate the person is in the process of standing up out of bed, etc. Doing so allows the tracking application 218 to monitor the patient's sleep times and duration, sleep cycles, periods of seated rest and/or lying rest, etc.

Because some pathologies (e.g., Lewy body dementia, etc.) are associated with more sleep and/or more rest, the tracking application 218 may diagnose these pathologies based on monitoring the person, e.g., to detect increased sleep and/or rest over time. Similarly, some pathologies restrict movement (or make movement more difficult), the tracking application 218 may diagnose these pathologies based on monitoring the person, e.g., to detect reduced activity levels, taking longer to complete certain movements (e.g., sitting, standing, etc.), etc. Further still, certain movements may be associated with pathologies. For example, restless leg syndrome may be associated with leg movements, which may be detected when a person is resting and/or sleeping. For example, using ankle-worn other wearables 110, the accelerometer 122 may capture these leg movements, and the tracking application 218 may determine the presence of these leg movements and determine the person has restless leg syndrome. As another example, tremors or other movements may be associated with Parkinson's disease. Therefore, the tracking application 218 may use data from the accelerometers 216, other sensors 204, and/or the other wearables 110, to detect tremors, and determine the patient has Parkinson's. As another example, the data from the accelerometers 216 (and/or the other sensors 204) may indicate the person periodically wakes up during sleep, e.g., to use the restroom, which may indicate the person has nocturia.

As another example, the tracking application 218 may analyze the data from the accelerometers 216 to determine the stability of the person's head, which may reflect balance. For example, poor head stability may indicate poor balance. Furthermore, the data from the accelerometers 122 of the other wearables 110 may indicate rapid, jerking movements, which may indicate poor stability and/or balance. Further still, a combination of these body parts may be used to determine an overall level of stability and/or balance. For example, if the head is largely stable but a smartwatch of the other wearables 110 indicates the person is frequently reaching their arms out to maintain balance, the tracking application 218 may determine the person has poor balance. Because poor balance is a symptom of some pathologies, the tracking application 218 may determine the person has such pathologies based at least in part on the monitored balance of the person.

In some embodiments, in addition to the brain wave detection discussed above, the tracking application 218 further computes the fatigue score based on the number of recorded eye blinks, the threshold number of eye blinks, eye blink durations, threshold eye blink durations, sleep patterns, rest patterns, tasks, and/or movements detected by the accelerometers 216. In some embodiments, the tracking application 218 recomputes the fatigue score over time and stores the fatigue score in the user profile 224. Doing so allows the tracking application 218 to monitor fatigue over time and generate recommendations accordingly.

More generally, the tracking application 218 functions as a comprehensive fatigue detection and cognitive performance monitoring system by integrating various sensors and analyzing the collected data to generate a fatigue score. As stated, the tracking application 218 receives input from electroencephalography (EEG) sensors to analyze brain waves for signs of fatigue or stress, monitors eye movements using eye-tracking sensors to evaluate blink frequency and duration, and employs the accelerometers 216 and/or 122 to measure reaction times in response to stimuli. Additionally, the tracking application 218 uses data from the accelerometers 216 and/or 122 to record the time taken to complete specific tasks or movements. The tracking application 218 uses data processing algorithms, including machine learning models 220, to identify patterns in the sensor data that correlate with fatigue indicators, such as slowed reaction times and altered blink metrics. By processing this information, the tracking application 218 computes a quantitative fatigue score and provides real-time feedback, which can alert users or connected systems to initiate appropriate interventions.

In some embodiments, the tracking application 218 may use sounds to detect patient reaction times. Generally, a sound may cause a reaction in other parts of the body, such as the head turning or arms moving in response to a startling sound. Therefore, the tracking application 218 may test the patient's reaction time by emitting sounds into the car via the speakers 206 and detecting the movements and/or reactions to the sound. If reactions are detected, attributes of the reactions (e.g., reaction time, type, etc.) may be stored in the user profile 224. Doing so allows the tracking application 218 to monitor the patient over time. For example, if the attributes of the reactions indicate the user is taking longer to react to sounds emitted via the speakers, the tracking application 218 may detect diminished reaction times. In such embodiments, the tracking application 218 may generate a notification and/or recommendation, e.g., to visit a doctor, prescribe hearing aids, etc. The notification and/or recommendation may be outputted via the earbuds 114a-114b, external devices 104 of the user, other wearables 110 of the user, etc. Doing so may be useful in detecting hearing loss and prescribing therapy, particularly in young patients such as babies who lack the ability to communicate their inability to hear.

The tracking application 218 may further monitor environmental factors over time, such as noise levels, light levels, crowdedness, temperature, etc. The earbuds 114a, 114b are equipped with advanced sensors 204 capable of tracking various environmental factors. For example, the speakers 206 may monitor ambient sound levels, providing insights into noise exposure, which may cause fatigue in people with certain pathologies. Additionally, the sensors 204 may measure surrounding light intensity, which may cause fatigue in people with certain pathologies. In some embodiments, the sensors 204 may include temperature sensors to detect changes in external temperature. Furthermore, the tracking application 218 may receive environmental data from external devices 104, such as motion sensors, thermostats, cameras, microphones, smartphones, devices on the Internet, etc. Therefore, the tracking application 218 may detect fatigue, mental stress, reductions in reaction time, etc., and correlate these detections with environmental conditions. Doing so may allow the tracking application 218 to diagnose certain pathologies. For example, a person demonstrating increased fatigue in a room with moderate ambient noise may have a pathology, whereas a person without the pathology may not encounter fatigue in the same room.

The tracking application 218 may further use the pulse oximeter 210 and/or other sensors 204, e.g., to determine oxygen saturation, detect movements, detect sounds, etc. For example, a person who is resting may have lower oxygen saturation levels as recorded by the pulse oximeter 210. The tracking application 218 may correlate the period of rest with the lower oxygen saturation levels in the user profile 224, and use the stored data for subsequent recommendations. For example, if the oxygen saturation levels are reduced, the tracking application 218 may output a notification to instruct the person to rest. The notification may be outputted via the earbuds 114a-114b, external devices 104 of the user, other wearables 110 of the user, etc.

Based on monitoring a patient, the tracking application 218 may generally determine one or more recommendations. In some embodiments, the recommendation may include detecting one or more pathologies of the person. The pathologies of the person may be specified in the user profile 224 and/or programmatically detected by the tracking application 218 as described herein. More generally, the tracking application 218 may detect movements by a patient, detect fatigue in the patient, detect reaction times of the patient, detect pathologies in the patient, detect therapy devices worn or otherwise used by the patient, predict pathologies and/or adverse health events of the patient, determine the position of sounds, track patient adherence to therapy, detect errors or configuration issues with therapy devices, and/or generate recommendations.

The user profiles 224 store a plurality of attributes for one or more users. For example, the user profiles 224 may store indications of movements detected by the tracking application 218 (with corresponding timestamps), electrical activity detected by the electrodes 214 (e.g., brain waves, eye blinks, etc.) data recorded by the sensors 204 (e.g., oxygen saturation values, respiratory rate, etc.), pathologies associated with the user, devices used by or otherwise prescribed to the user (e.g., RPT device 106, a mask 108, etc.), use of the devices prescribed to the user (e.g., a log of entries detailing dates and times when the user uses their mask 108, RPT device 106, etc.), detected apneas (which may be associated with a timestamp and a detected sleep position when the apneas occur), detected hypopneas (which may be associated with a timestamp and a detected sleep position when the hypopneas occur), or any other attribute of the user. The user profiles 224 may further include demographic information for a plurality of users (or groups of users), such as average reaction times, average sleep times, average movement speed, average blinks, etc. Doing so may allow the tracking application 218 to compare data of a monitored patient to the demographics to identify pathologies, predict adverse health events, etc.

The device profiles 226 include data describing different devices, such as RPT devices 106, masks 108, other wearables 110, external devices 104, etc. Example attributes stored in the device profiles 226 include device type, device model, device function, sound profiles, how the device is worn or otherwise used by the patient, configurations, associated components, and the like. The device profiles 226 may further include associations between devices (e.g., RPT devices 106, masks 108, other wearables 110, etc.) and one or more pathologies for which the devices are prescribed to provide therapy. The device profiles 226 may further include indications of sleep positions that negatively impact a pathology and/or the ability of a device to deliver therapy.

As stated, the accelerometer 216 (which is representative of the accelerometers 122 of the other wearables 110) is a device that measures the rate of change of velocity (e.g., acceleration) of the earbud 114a along three orthogonal axes (X, Y, and Z, in three-dimensional space), providing data on movement and orientation of the earbud 114a. The data provided by the accelerometer 216 may therefore be acceleration data in units of meters per second squared (m/s²) (or “g”, where 1 g is approximately 9.8 m/s², the acceleration due to the Earth's gravity). In some embodiments, the accelerometer 216 provides data reflecting static forces (e.g., the pull of gravity when the earbud 114a is stationary, which may be used to determine tilt and/or orientation) and dynamic forces (e.g., forces from motion or vibration, which may be used to detect movement patterns).

The tracking application 218 executing on processor 116e of earbud 114a may use the acceleration data from the accelerometer 216 to determine position, e.g., by computing a first integration of the acceleration data over time to determine velocity. The tracking application 218 may compute a second integration of the velocity to determine the position of the earbud 114a. Therefore, using the data from the accelerometer 216, the tracking application 218 detects (or determines) acceleration, velocity, and/or movement.

To determine the position of a person wearing the earbuds 114a, 114b, the tracking application 218 may process the sensor data along each of the X, Y, and Z axes from the accelerometer 216 over one or more time intervals (e.g., milliseconds, seconds, minutes, etc.). In some embodiments, a baseline calibration is performed using the accelerometer 122, e.g., to determine the orientation when the person is standing upright, sitting, etc., to calibrate the X, Y, and Z axes relative to gravity.

For example, if the sensor axis pointing upward shows a dominant gravitational pull (e.g., the Z-axis is approximately 9.8 m/s², and the X and Y axes are approximately zero), the tracking application 218 may determine the person is lying on their back (e.g., in a supine position). As another example, if the sensor axis pointing downward shows a dominant gravitational pull (e.g., the Z-axis is approximately −9.8 m/s², and the X and Y axes are approximately zero), the tracking application 218 may determine the person is lying on their stomach (e.g., in a prone position). Further still, if the data from the accelerometer 122 reflects minimal acceleration on the Z-axis but a strong signal on the X-axis (or Y-axis, depending on the assignment of axes), the tracking application 218 may determine the person is lying on their side. The particular side that the person is lying on may be based on the detected forces. For example, if the X-axis is approximately 9.8 m/s², and the Y and Z axes are approximately zero, the tracking application 218 determine the person is lying on their right side. As another example, if the X-axis is approximately −9.8 m/s², and the Y and Z axes are approximately zero, the tracking application 218 may determine the person is lying on their left side. In some embodiments, when the tracking application 218 determines a person is lying down, the tracking application 218 may further determine whether the person is sleeping, e.g., based on electrical signals captured by the electrodes 214, snoring noises detected by the microphone arrays 208, noises generated by the RPT device 106, etc.

Furthermore, the tracking application 218 may use the accelerometers 216 to detect fatigue and/or the onset of sleep. For example, the tracking application 218 may detect subtle movements such as head tilting via the accelerometers 216 (e.g., subtle forward or side ward tilt of the head at specific angles (e.g., 10-20 degrees) as muscles relax), reduced movement (e.g., decreasing frequency and amplitude of voluntary head movement for a predetermined period of time such as 5-10 seconds), micro movements (e.g., small, erratic movements followed by stillness), consistent posture (e.g., the head remains in a fixed position for an extended period), etc. For example, data from the accelerometer 216 may indicate characteristic patterns of nodding (e.g., periodic head dips), jerky movements followed by stillness, a reduction in high frequency movements (e.g., talking, walking, eating, etc.), etc.

Similarly, the processors 116b of the other wearables 110 may compute the first and second integrations based on the acceleration data from the accelerometers 122 to determine the respective positions of the other wearables 110. Further still, the processors 116b of the other wearables 110 may perform the same X, Y, and Z axis processing to determine whether the corresponding body part is facing up, down, or to the side (and to which side based on the particular configuration). However, in some embodiments, the raw sensor data from the accelerometers 122 of the other wearables 110 may be transmitted to the tracking application 218, which may determine the position of the other wearables 110, and the associated body part, as described above.

In some embodiments, the tracking application 218 may classify data collected by the earbuds 114a-114b and/or other wearables 110 as being associated with a particular activity (e.g., walking, running, sleeping, standing, sitting, etc.). For example, data from the accelerometers 216 may reflect peaks and troughs corresponding to steps while walking or running. As another example, the accelerometers 216 may capture rotation information that reflects movement during activities such as yoga. Therefore, the tracking application 218 may classify any number and type of activities based on data collected by the earbuds 114a-114b and/or other wearables 110. Further still, by identifying a particular activity, the tracking application 218 may store metadata for each activity, such as time to completion, number of instances, etc. Doing so may allow the tracking application 218 to detect a change in reaction time. For example, if the data in the user profile 224 indicates the average amount of time a person needs to stand up has increased by 2 seconds in the last 3 months, the tracking application 218 may determine a negative change. The tracking application 218 may then use the data to identify pathologies and/or treatments for the pathologies, e.g., in the therapies 222.

As stated, the tracking application 218 may further consider data from the accelerometers 122 of the other wearables 110 to detect fatigue and/or the onset of sleep. For example, if a smartwatch indicates rapid hand movements (e.g., sudden, rapid movements of the steering wheel) at a time when the earbuds detect the person rapidly raising their head (e.g., when they are waking from sleep), the tracking application 218 may determine the person is falling asleep while driving. Doing so may allow the tracking application 218 to generate an alert, engage safety features in the vehicle, etc.

As another example, the tracking application 218 may detect other activities, such as standing from a seated position, sitting from a standing position, getting out of bed, getting into bed, etc. For example, the tracking application 218 may use data from the accelerometer 216 (and/or accelerometers 122) to analyze the sequence of operations when a person stands from a seated position, characterized by changes along the X, Y, and Z axes of the accelerometers 216. Initially, during stillness, the Z-axis may reflect a stable gravitational force due to the seated posture, while the X and Y axes show minimal variation unless there is slight shifting. As the person leans forward in preparation for standing, the Z-axis magnitude decreases due to the forward tilt, reducing the vertical gravitational component, while either the X or Y axis increases depending on the leaning direction. When pushing off and rising, the Z-axis registers a pronounced increase in positive acceleration countering gravity, accompanied by variations in the X and Y axes capturing lateral or forward motion, which vary based on the standing technique. Upon reaching an upright position, the Z-axis stabilizes near 1 g, indicating alignment with gravity, while the X and Y axes return to near-zero or low values, signaling minimal horizontal movement. Optional transitional movements such as slight swaying or adjustments to stabilize balance may introduce small fluctuations across all axes after standing.

Since the earbuds 114a, 114b are worn in the cars of the person, in some embodiments, the position and/or movement determined by the tracking application 218 based on the data from the accelerometers 216 may be associated with the person's head. As stated, in some embodiments, the position and/or movement may be determined based on data from the other wearables 110. For example, in some embodiments, the tracking application 218 may receive the position data from the other wearables 110 and base the determination of a sleep position based on the received data.

As another example, if the other wearables 110 include a chest strap monitor worn on the person's chest, the processor 116b may use data from the accelerometer 122 to determine the orientation of the chest strap monitor. Because the chest strap monitor is worn on the chest, the data from the accelerometer 122 therefore indicates the orientation of the chest. Therefore, the chest strap monitor may provide the orientation data (and/or the raw sensor data from the accelerometer 122) to the tracking application 218 of the earbud 114a via the network 112. The tracking application 218 may therefore further determine the position and/or movements of the person based on the data from the chest strap monitor (and chest or torso by association).

For example, if the tracking application 218 determines the person is sleeping in a supine position, while the chest strap monitor indicates the chest is pointing upward (e.g., the accelerometer 122 data from the chest strap monitor indicates the Z-axis is approximately 9.8 m/s², and the X and Y axes are approximately zero), the tracking application 218 may determine (to a greater degree of confidence) that the person is sleeping on their back.

However, in some embodiments, a position and/or movement is multi-modal, e.g., reflects the position and/or movement of different body parts. Therefore, the tracking application 218 may collect data from the other wearables 110 to determine the orientation of the associated part of the body the other wearables 110 are worn on or otherwise proximate to. Continuing with the previous example, the tracking application 218 may determine the person's head is facing upward (e.g., in the supine position) and the torso is facing upward (e.g., in the supine position).

Similar determinations may be made for other wearables 110, e.g., for smartwatches worn by the person, devices worn on the legs (e.g., ankle step trackers, etc.), devices worn on the hips, etc. Generally, the accelerometer 122 of any of the other wearables 110 may provide data used to determine a position and/or movement of the device (and body part, by association) as described herein. For example, if the data from the earbuds 114a-114b, smartwatch, leg-worn devices indicate the person is sleeping in a combination of sleep positions (e.g., head in a first orientation, torso in a second orientation, legs, in a third orientation, etc.), which may result in poor posture of the spine, the tracking application 218 may generate an alert to cause the person to change sleep position.

As another example, the tracking application 218 may determine, based on the data from the accelerometer 216, that the forces are in the X or Y axes, with minimal or no forces in the Z position, indicating the face is turned to the side (e.g., one side of the face against the pillow or bed). Furthermore, the chest strap monitor may indicate the torso is facing down (e.g., forces are negative on the Z axis, with minimal or no forces on the X or Y axes). Therefore, based on these determinations, the tracking application 218 may determine the person is sleeping on their stomach.

More generally, using the disclosed techniques, the tracking application 218 may continuously monitor the position and/or movements of the patient, e.g., at predetermined time intervals. Doing so may advantageously detect positions and/or movements that may negatively impact the person's health. In response, the tracking application 218 may generate one or more recommendations. In some embodiments, the tracking application 218 references the therapies 222, which includes associations between one or more positions and/or movements and one or more pathologies, therapies, and/or treatments. For example, the therapies 222 may indicate, for a person falling asleep while driving, to output sounds via the speakers 206 and/or vibrations via the haptic feedback modules 212 to wake the person. Doing so may cause the person to wake up and stop driving. In some embodiments, the recommendations may include audible instructions outputted by the speakers 206, e.g., spoken words instructing the person to stop driving.

In some embodiments, the tracking application 218 determines one or more pathologies of the patient, e.g., based on the user profiles 224, the microphone arrays 208 detecting sounds associated with therapy devices such as RPT device 106 and/or masks 108, etc. The tracking application 218 may therefore analyze the positions, movements, and/or the identified pathologies to determine the person's health may be negatively affected. For example, if the person has positional sleep apnea associated with a particular sleep position, and the tracking application 218 determines that the person is sleeping in that particular position, the tracking application 218 may generate one or more recommendations. For example, the therapies 222 may indicate to output sounds via the speakers 206 and/or vibrations via the haptic feedback modules 212 to wake the person. Doing so may cause the person to change sleep positions.

As another example, if the person has obstructive sleep apnea (OSA), sleeping on the back may exacerbate apneas and/or hypopneas. Therefore, based on a determination that the person is sleeping at least partially on their back (e.g., the head and/or torso are facing upward) and has OSA, the tracking application 218 may determine to generate a recommendation. Therefore, in some embodiments, the therapies 222 includes associations between one or more positions and/or activities, one or more pathologies, and one or more therapies or treatments. For example, the therapies 222 may specify, for the OSA patient sleeping on their back, to modify the therapy provided by the RPT device 106. The modification of the therapy may include modifying pressure, changing pressure mode (e.g., fixed pressure mode and/or auto-adjusting (APAP) pressure mode), ramp time, pressure titration, humidification, tidal volume, respiratory rate, inspiratory time, rise time, etc. In some embodiments, the tracking application 218 may transmit an instruction to the RPT device 106 to implement the therapy modifications identified in the therapies 222 in real-time. In some embodiments, the tracking application 218 may transmit an indication of the modifications to the patient's medical provider, e.g., to change the person's prescription and modify the RPT device 106 accordingly.

More generally, the tracking application 218 may generate any number and type of recommendations. For example, the recommendations may include changing the type of mask 108 used by the patient, providing educational recommendations such as using fewer (or more) pillows, changing mattress type (e.g., a firmer mattress, a softer mattress, etc.), displaying one or more pages of the instruction manual for the RPT device 106 and/or mask 108 on the user's smartphone to assist the user to properly wear the devices, etc.

In some embodiments, the data from the accelerometers 216 may be used by the tracking application 218 to determine an angle of inclination of a body part as part of the position and/or activity (e.g., that the person's head is nodding forward at a x-degree angle). In some embodiments, accelerometer 216 calibration may occur when the person is in a particular position (e.g., lying flat, sitting upright, etc.), which provides the tracking application 218 a reference for a neutral position. The gravitational force vector components (aₓ, a_{y}, a_{z}) from the accelerometer 216 may be used to compute the tilt angles (e.g., pitch for front to back tilt, roll for side to side tilt) of the earbud 114a, 114b. For example, pitch angle θ may be computed according to the following equation:

$$θ = arc tan ( \frac{{a}_{x}}{\sqrt{{a}_{y}^{2}} + {a}_{z}^{2}} ) .$$

Similarly, roll ϕ may be computed according to the following equation:

$$ϕ = arctan ( \frac{{a}_{y}}{\sqrt{{a}_{x}^{2} + {a}_{z}^{2}}} ) .$$

Therefore, the tracking application 218 may use the determined angles to provide recommendations. For example, a person may be considered to be falling asleep when in a seated position and having a head tilt angle such as 10 degrees. Therefore, if the tracking application 218 subsequently determines the person is seated and has a head tilt angle greater of approximately 10 degrees, the tracking application 218 may determine the person is falling asleep while driving and generate a recommendation. For example, the recommendation may wake the person, initiate autonomous driving, etc., to prevent an accident.

In some embodiments, the tracking application 218 may predict events based on monitoring a patient. For example, if a person has difficulty walking to the bathroom without assistance, the tracking application 218 may predict that a fall event may occur before the person gets up to go to the bathroom. The tracking application 218 may therefore generate an audible alert or vibration to cause the patient to use a cane.

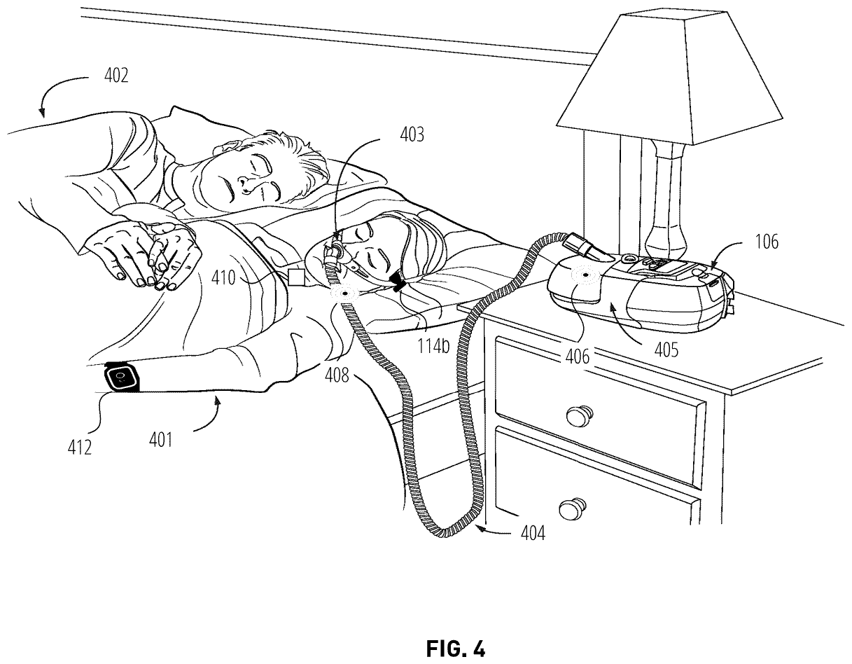

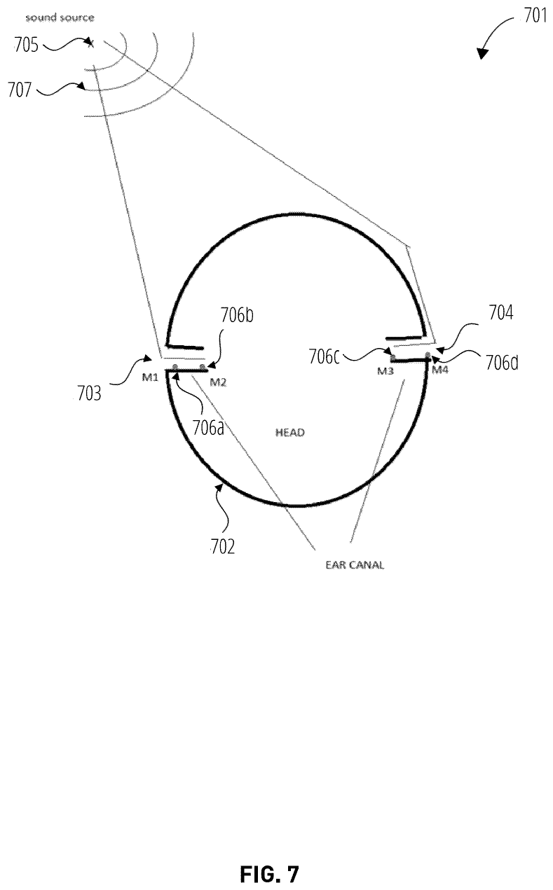

In some embodiments, the tracking application 218 uses sound analysis for patient monitoring, reaction time tracking, and fatigue and/or sleep detection. Generally, the microphone arrays 208 of an earbud 114a, 114b may capture sounds and determine the type and location of the sound. Examples of such sounds include soundwaves 406, 408, and 707, of FIG. 4 and FIG. 7, respectively. For example, the tracking application 218 may detect sounds associated with driving (e.g., noises made by vehicles), detect sounds associated with airway collapses in an airway of the person, detect therapy devices such as RPT device 106 and/or mask 108, etc.

Therefore, in some embodiments, the tracking application 218 may detect apneas or hypopneas while a person is sleeping, e.g., based on detecting sounds associated with apneas and/or hypopneas. The tracking application 218 may determine the sleep position of the patient when the apneas or hypopneas are detected. The tracking application 218 may associate each detected apnea or hypopnea with the corresponding sleep position in an entry of the user profile 224 of the person. As stated, the sleep position may include the positions of multiple body parts and/or an angle of inclination. Doing so allows the tracking application 218 to predict events, e.g., apneas or hypopneas, based on the current sleep position and the data in the user profile 224. For example, if the user profile 224 indicates the person has hypopneas that exceed a threshold while sleeping on their stomach, the tracking application 218 may generate an alert when determining the person is sleeping on their stomach.

As another example, the tracking application 218 may analyze sounds captured by the microphone arrays 208 and determine the sounds are associated with a partial airway obstruction (e.g., a hypopnea). The tracking application 218 may determine the sleep position of the person when the hypopnea is detected (e.g., the person is sleeping on their back). Based on the detection of the hypopnea and the sleep position, the tracking application 218 may generate a recommendation. For example, the tracking application 218 may output a notification, alert, sound, vibration, etc., to cause the person to change sleep positions. The notification may be outputted via the earbuds 114a-114b. In some embodiments, the tracking application 218 causes the notification to be outputted on the external devices 104 (e.g., the user's smartphone), the other wearables 110 (e.g., on the user's smartwatch), the RPT devices 106, and/or masks 108.

In some embodiments, the tracking application 218 may determine that the sleep position of the person is interfering with the delivery of respiratory therapy by the RPT device 106 and/or mask 108. For example, by analyzing soundwaves detected by the microphone arrays 208, the tracking application 218 may detect an airflow obstruction that is at least partially obstructing the flow of air to the person. For example, a mask 108 may have one or more conduits, or tubes, which deliver pressurized air therapy to the person. The sleep position of a patient may cause these tubes to be obstructed, e.g., when the person's sleep position impinges or otherwise restricts the flow of air through the tubes. As such, the tracking application 218 may output an indication to cause the patient to change sleep positions, e.g., by outputting sounds and/or vibrations.

As another example, the tracking application 218 may recommend a different mask 108 for the patient based on the sleep position and/or sleep movements by the patient. For example, by monitoring sounds, the tracking application 218 may detect an air leak in the mask 108 used by the patient when the patient is sleeping in a particular position. For example, by detecting a sound that matches a stored sound of an air leak and determining the patient is sleeping on their side, the tracking application 218 may recommend a different type of mask 108 that is more suitable for side sleeping, a mask 108 that is more suitable for significant movements during sleep, etc.

In some embodiments, the tracking application 218 uses additional information collected by the sensors 204 to determine a sleep position. For example, the pulse oximeter 210 may record oxygen saturation (SpO₂) values from the bloodstream of the patient at predetermined intervals (and/or based on instructions from the tracking application 218, e.g., when the tracking application 218 detects a sleep position, a sound, detects a therapy device, detects a pathology, etc.). In some embodiments, the oxygen saturation values are stored in the user profile 224 of the patient. The tracking application 218 may use the oxygen saturation values to determine, at least in part, a sleep position of the patient. For example, if the patient's oxygen saturation is below a threshold, the tracking application 218 may determine that the patient is sleeping on their back (e.g., because the patient is not experiencing suitable SpO₂ levels). The thresholds may be any type of threshold, such as predetermined minimum/maximum oxygen saturation thresholds, thresholds that are associated with a specific patient (e.g., patient's average oxygen saturation while sleeping on their back, side, or stomach, etc.), thresholds associated with a group of patients (e.g., patients with a specific type of sleep position, pathology, etc.). In some embodiments, the thresholds are stored in a patient's user profile 224.

The tracking application 218 and/or models 220 may generally use any location (or position) determination algorithm to determine the location where a sound detected by the microphone arrays 208 originated. Because multiple microphone arrays 208 are included in an earbud pair 102 (whether in a single earbud 114a, 114b or across both earbuds 114a, 114b), positions may be determined using measurements from these fixed points to compute the precise location a sound originated, e.g., using algorithms such as triangulation, trilateration, beamforming, single or multiple microphone acoustic impedance measurements, impulse/frequency response function measurements, etc.

For example, when the plurality of microphone arrays 208 detect a sound, the tracking application 218 and/or the models 220 may receive indications of the sounds (e.g., waveforms) from the microphone arrays 208. The tracking application 218 and/or the models 220 may determine a position of the sound source by measuring the time difference of arrival (TDOA) of the sounds experienced by the microphone arrays 208. As another example, the tracking application 218 and/or the models 220 may perform a mathematical cross-correlation operation that measures the similarity between two detected signals as a function of the time-lag applied to one of them. By shifting one signal in time and calculating the correlation at each shift, the cross-correlation allows the tracking application 218 and/or the models 220 to identify the time offset that maximizes the similarity between the two signals. Although discussed with reference to the tracking application 218 and/or the models 220, the microphone arrays 208 may include logic to perform the position and/or location determination described herein. Similarly, the external devices 104 may include instances of the tracking application 218, models 220, therapies 222, and user profiles 224, e.g., to perform the processing described herein (e.g., based on receiving data collected by the earbuds 114a, 114b).

Furthermore, the microphone arrays 208, the processor 116e, the tracking application 218, and/or the models 220 may analyze the sounds to identify one or more attributes of the detected sounds (e.g., pressure, amplitude, wavelength, and/or frequency). Doing so may be useful to identify the sounds (and/or causes thereof), e.g., sounds associated with movements, sounds associated with therapy devices, sounds associated with pathologies, airway obstructions, etc., as described herein. For example, the pressure, amplitude, wavelength, and/or frequency may be compared to one or more known sounds, e.g., to identify a known sound that is similar to the detected sound. As stated, the known sounds may be stored or otherwise reflected in the models 220 and/or the device profiles 226. Similarly, the models 220 may consider the attributes of the sound and return a known sound as being similar to the detected sounds. The known sounds may be stored by the tracking application 218, e.g., in the device profiles 226. Similarly, the known sounds and/or attributes thereof (e.g., pressure, amplitude, wavelength, frequency, etc.) may be stored as features in the models 220. Doing so allows the tracking application 218 and/or models 220 to match a detected sound to a known sound (e.g., based on one or more of pressure, amplitude, wavelength, frequency, etc.). For example, the tracking application 218 may analyze a sound and determine the sound is associated with an airway obstruction. Doing so allows the tracking application 218 to generate a recommendation, e.g., based on the detected airway obstruction and the sleep position of the person.

In some embodiments, a device may be associated as a treatment for one or more pathologies in the device profiles 226. As such, the tracking application 218 and/or models 220 may identify a pathology associated with a detected device and/or sound. For example, the tracking application 218 and/or models 220 may receive a detected sound as input (including any attributes thereof). The tracking application 218 and/or models 220 may identify a known sound, such as the sounds made by an RPT device 106 that are stored in the device profiles 226 and/or models 220, and determine a patient is using the RPT device 106. The tracking application 218 and/or models 220 may identify a corresponding pathology based on the detected device, e.g., RPT device 106. The tracking application 218 and/or models 220 may further identify a therapy 222 associated with the detected device and/or pathology, such as changing one or more parameters of the RPT device 106 providing therapy to the patient.

In some embodiments, distances between respective pairs of the microphone arrays 208 in a given earbud 114a are stored in the memory 118e (e.g., in the tracking application 218, the models 220, etc.) to facilitate the detection of pathologies in a patient, e.g., for use in a location detection algorithm. Similarly, the distance between earbud 114a and earbud 114b may be determined at predetermined time intervals such that both earbuds 114a, 114b, can be used to detect sounds (e.g., associated with devices worn by the patient and/or pathologies of the patient) over time. For example, the processor 116e of earbud 114a may cause the communications interface 120c of the earbud 114a to emit one or more radio signals to earbud 114b. The processor of earbud 114b may determine the distance to earbud 114a based on the radio signals (and any data included in the signals), e.g., based on one or more of received signal strength (RSS), time of flight (ToF), and angle of arrival (AoA). The earbud 114b may return an indication of the determined distance to earbud 114a via the communications interface 120c. More generally, technique may be used to determine distances between the earbuds 114a and 114b in a given earbud pair 102. Once determined, the distances between the earbuds 114a, 114b (as well as between two or more microphone arrays 208, as these distances are fixed) can be used as points in space to determine the position a sound originated.

Because the microphone arrays 208 are placed at known locations, the tracking application 218 and/or the models 220 may use these (or other) algorithms to calculate the exact position of the source of the sound. Doing so may allow the tracking application 218 to determine activities and/or movements of a patient, detect therapy devices being worn or otherwise used by the patient, detect pathologies, detect airway obstructions, etc. As described herein, the distances between the earbuds 114a, 114b (and the corresponding microphone arrays 208) may be periodically determined, e.g., to facilitate the detection of a movement or activity by the patient, detection of a pathology using both earbuds 114a, 114b, determination of a sleep position of the patient, detection of a device such as RPT device 106 or mask 108 used by the patient, etc. Furthermore, using the models 220, the tracking application 218 and/or the models 220 may adjust the position determination algorithm to compensate for how sounds travel through the airway, how sounds travel through the ear canal, how sounds travel through fluid, how sounds travel through tissue, etc. Doing so allows the tracking application 218 and/or the models 220 to accurately determine the location where a sound originated.

In some embodiments, the tracking application 218 may detect the location of an airway obstruction in the patient based on sounds generated as the airway closes. In some embodiments, the tracking application 218 may determine a degree of the obstruction, e.g., partial, complete, etc. In some embodiments, the tracking application 218 may determine a type of the obstruction based on the sounds, e.g., OSA, positional sleep apnea, etc. In some embodiments, positional sleep apnea is determined based on position information received from the accelerometer 216 and/or one or more other wearables 110, e.g., to determine whether the patient is sleeping while seated upright in a chair, etc.

In some embodiments the tracking application 218 and/or the models 220 may determine whether the determined location is within the body of the patient, e.g., to identify external sounds. For example, the tracking application 218 and/or the models 220 may determine whether at least a portion of the sound was captured by the microphone arrays 208 through the body, e.g., through the airways, through the ear canal, through the tissues of the body, etc. If the tracking application 218 and/or the models 220 determine the sound was captured by the microphone arrays 208 through the body, the tracking application 218 and/or the models 220 may determine that the sound originated from within the body. In such an example, the tracking application 218/or the models 220 may exclude external therapy devices such as RPT device 106 as being the source of the sound and instead consider internal therapy devices as the source of the sound. Similarly, if the tracking application 218 and/or the models 220 may determine that the sound originated external to the body, the tracking application 218 may determine that the person is in a room with ambient noise (e.g., with people talking, watching a movie, listening to music, etc.).

In addition and/or alternatively, the tracking application 218 and/or the models 220 may consider the distance to the determined sound location relative to one or more of the earbuds 114a, 114b. For example, if the distance between the determined sound location and one or more of the earbuds 114a, 114b is 10 meters, the tracking application 218 and/or the models 220 may determine that the sound did not originate from within the body, as this distance is too great to originate from within the body. In addition and/or alternatively, the tracking application 218 and/or the models 220 may consider the direction of the sound captured by the microphone arrays 208. For example, if the direction of the sound indicates the sound was generated above and behind the earbuds 114a, 114b, the tracking application 218 and/or the models 220 may determine that the sound did not originate from within the body. The accelerometer 216 may provide position information to facilitate the direction from which the sound originated relative to the body, e.g., the head and/or ears of the body. Doing so may allow the tracking application 218 to determine specific therapy devices worn or otherwise used by the patient. For example, based at least in part on a determination that a sound originated near the patient's face, the tracking application 218 may determine the sound is associated with a mask 108 being worn by the patient. As another example, based at least in part on a determination that the sound originated a meter away from the patient, the tracking application 218 may determine the sound is associated with an RPT device 106 used to deliver therapy to the patient. Embodiments are not limited in these contexts.

More generally, the tracking application 218 and/or the models 220 may analyze one or more of the sounds detected by the plurality of microphone arrays 208. For example, the analysis may be used to determine a position and/or movement of the patient, determine a therapy device worn by the patient, determine a pathology associated with the sounds and/or device, determine errors or other problems with therapy devices such as RPT devices 106 and/or masks 108, etc. For example, the tracking application 218 and/or the models 220 may perform waveform analysis on the soundwaves detected by the microphone arrays 208. For example, the tracking application 218 may compare the soundwaves (e.g., the waveforms, attributes of the soundwaves, etc.) to known examples of types of sounds, e.g., sounds associated with devices, sounds associated with airflow leaks (e.g., in masks 108 and/or RPT devices 106), sounds associated with airflow obstructions (e.g., in masks 108 and/or RPT devices 106), sounds associated with pathologies, etc. In some embodiments, the known types of sounds and associated sleep positions, pathologies, and/or associated devices may be stored in the tracking application 218, e.g., in the device profiles 226. As another example, the sounds associated with a particular sleep position may be stored in the user profiles 224. Therefore, if a sound (e.g., the sound of an airway obstruction) is similar to a stored sound associated with back sleeping in the user profile 224 of the person, the tracking application 218 may determine the person is sleeping on their back at least partially based on the sound.

As another example, if a sound is similar to a stored sound in the device profile 226 associated an RPT device 106, the tracking application 218 and/or the models 220 may determine the patient is using an RPT device 106, which may be associated with OSA. As such, the tracking application 218 and/or models 220 may determine the patient has OSA. As another example, if a sound is similar to a stored sound associated with a therapy device such as RPT device 106 or mask 108, the tracking application 218 and/or models 220 may determine that the patient is wearing or otherwise using the therapy device.

As stated, in some embodiments, the tracking application 218 determines an activity, a movement, a position, device, a pathology, a location of the pathology, and/or any attribute thereof based at least in part on the models 220. The models 220 represent any type of model, such as a machine learning (ML) model, neural network, large language model (LLM), or any other type of artificial intelligence (AI) model. For example, the models 220 may include models trained to identify locations of input sounds, models trained to identify sounds based on input sounds (e.g., sounds associated with particular positions, sounds associated with particular activities and/or movements, sounds of obstructions at a plurality of points in the airway, sounds of types of obstructions, sounds associated with therapy devices, sounds generated by other parts of the human body, etc.), models of the airway, models of the ear canal, models trained to determine how sounds travel through the airway, models trained to determine how sounds travel through the ear canal, models trained to determine how sound travels through tissues, models trained to determine how sounds travel through fluid, and models trained to generate treatments or other recommendations for identified movements, positions, reaction times, sleep states, pathologies, etc.

In artificial intelligence embodiments, the models 220 may be trained based on training data, e.g., data describing different sounds (and/or soundwaves) associated with positions, activities, or movements, data from a plurality of users, data describing therapies for pathologies, etc. For example, the training data may include sounds (and/or soundwaves), attributes of the sounds and/or soundwaves, etc. The models 220 may be trained to identify features of the sounds such that once trained, the models 220 may return a sound similar to an input sound. For example, the models 220 may be trained to identify the sounds of a person falling. Based on an input sound of a person falling, the models 220 may determine that the input sound is similar to the sound of a person falling. Similarly, the models 220 may be trained to identify other data associated with input sounds, such as associated positions, activities, pathologies, therapy devices, treatments, etc. Therefore, the models 220 may return a movement associated with the input sound, a pathology associated with the sound, a therapy associated with the pathology, etc. The models 220 may be retrained over time, e.g., to be tailored to a particular user, position, movement, device, and/or pathology. Embodiments are not limited in these contexts.

In some embodiments, the earbuds 114a, 114b may operate according to two or more detection modes. For example, a first mode may include the earbuds 114a, 114b monitoring and analyzing sounds as described above. In such an example, a second mode may include the earbuds 114a, 114b periodically emitting sounds into the ear canal via the speaker 206 and/or vibrations into the ear canal via the haptic feedback module 212. The microphone arrays 208 and/or haptic feedback module 212 may detect response signals (e.g., reflections) from these sounds and/or vibrations. The tracking application 218 may then use triangulation, beamforming, trilateration, single or multiple microphone acoustic impedance measurements, impulse/frequency response function measurements, or any suitable location detection algorithm to detect a position, an activity, a movement, a therapy device, an airway obstruction, pathology, or any attribute thereof. A third mode of operation may include using electrodes 214 and/or accelerometers 216 (or any other component of the earbuds 114a-114b) to track patient reaction times, levels of fatigue, and/or sleep states. A fourth mode of operation may include detecting the movements of a person. A fifth mode of operation may include any combination of the first, second, third, and fourth modes of operation.

In some embodiments, the tracking application 218 may cause the earbuds 114a, 114b to change between the different modes of operation. The tracking application 218 may change the modes of operations according to any number and type of criteria. For example, the tracking application 218 may change the mode of operation at predetermined time intervals. In addition and/or alternatively, the tracking application 218 may change the mode of operation based on attributes of the waveform of a detected sound (e.g., pressure, amplitude, wavelength, frequency, etc.). For example, if the electrodes 214 detect brain activity reflective of fatigue and/or the onset of sleep, the tracking application 218 may change the mode of operation of the earbuds 114a, 114b, e.g., to monitor the patient and generate recommendations as described herein. Embodiments are not limited in these contexts.

In some embodiments, the external devices 104, RPT devices 106, other wearables 110 and/or masks 108 include instances of the tracking application 218, not pictured in FIG. 1 for the sake of clarity. In such embodiments, the instances of the tracking application 218 on the earbuds 114a-114b communicate with the instances of the tracking application 218 on the external devices 104, RPT devices 106, other wearables 110, and/or masks 108 (e.g., to transmit notifications, recommendations, tasks, goals, or any other type of content). In some embodiments, the tracking applications 218 may transmit notifications via an operating system (not pictured) of the external devices 104, RPT devices 106, other wearables 110 and/or masks 108.

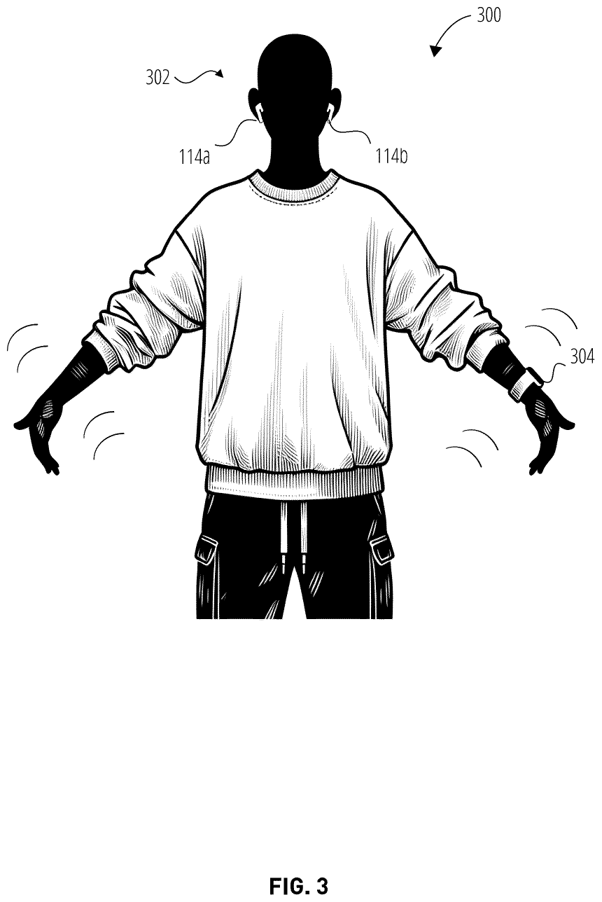

FIG. 3 is a schematic 300 depicting a person 302 wearing earbuds 114a, 114b, and a smartwatch 304 (e.g., one of the other wearables 110). The schematic 300 depicts that the person is standing. The tracking application 218 may generally determine the person 302 is standing based on data from the accelerometers 216 of the earbuds 114a, 114b and/or the accelerometer 122 of smartwatch 304.

Furthermore, the tracking application 218 may generally determine the balance of the person 302, e.g., based on data from the accelerometers 216 of the earbuds 114a, 114b and/or the accelerometer 122 of smartwatch 304. For example, the data from accelerometers 216 of earbuds 114a, 114b, indicate the person's head is relatively stable. Therefore, in such embodiments, the tracking application 218 may determine the person has adequate stability and/or balance. In some embodiments, the data from the accelerometer 122 of smartwatch 304 may indicate a lack of stability, e.g., because the arms are separated from the body in a motion that reflects a lack of balance. Therefore, in such embodiments, the tracking application 218 may determine the person is having difficulty maintaining balance and/or stability. As such, the tracking application 218 may determine the person has a pathology that has balance problems as a symptom. Further still, in some embodiments, the tracking application 218 may detect adverse health events. For example, based on determining the person 302 lacks balance, the tracking application 218 may predict that the person 302 may fall while walking. As such, the tracking application 218 may output a notification to use a cane, sit down, call for assistance, etc. The notification may be outputted via the earbuds 114a-114b, external devices 104 of the user, other wearables 110 of the user, etc. For example, the tracking application 218 of earbuds 114a-114b may transmit an instruction or other indication of the notification to the external devices 104 of the user and/or other wearables 110 of the user, e.g., via the network 112.

As stated, the tracking application 218 may monitor the movements of the person 302 over time. Therefore, the schematic 300 may depict the person 302 during a single point in time of an overall movement. For example, the person 302 may be standing from a seated position, or bracing themselves to sit down or lie down. By tracking the movements of the person 302, the tracking application 218 may determine how long the person 302 needs to complete a movement, which may be stored in the user profile 224 for the person 302. Over time, the tracking application 218 may detect the person making similar movements, and detect changes in the amount of time required to make the movements. For example, the tracking application 218 may determine that the average time the person 302 requires to stand up or sit down has increased by 3 seconds in a 2-month period. In such an embodiment, the tracking application 218 may generate a recommendation, e.g., to use a cane, rest, visit their medical provider, etc.

FIG. 4 shows a system including a patient 401 wearing a patient interface 403, in the form of nasal pillows, receiving a supply of air at positive pressure from an RPT device 106. The patient interface 403 represents the mask 108. Air from the RPT device 106 is humidified in a humidifier 405, and passes along an air circuit 404 to the patient 401. A bed partner 402 is also shown. The patient interface 403 is one example of a patient interface, or mask 108. Other examples include, but are not limited to, a nasal mask, a full-face mask, etc. As shown, the patient 401 is wearing earbud 114a (and corresponding earbud 114b, which is not depicted). In one or more embodiments, the earbuds, patient interface 403, RPT device 106, and humidifier 405 form a respiratory therapy system for treating a respiratory disorder.

As shown, the patient 401 is wearing a chest monitor 410, which may include heartrate monitoring capabilities among other biometric tracking capabilities. Similarly, the patient 401 is wearing a smartwatch 412, which may include various biometric tracking capabilities, e.g., heartrate tracking, oxygen saturation tracking using a pulse oximeter, stress tracking, sleep tracking, etc. The chest monitor 410 and smartwatch 412 represent the other wearables 110 and smartwatch 304. Therefore, the chest monitor 410, smartwatch 304, and smartwatch 412 each include a respective processor 116b, memory 118b, communications interface 120b, and accelerometer 122.

As stated, the tracking application 218 of the earbuds 114a, 114b may determine the position and/or activities of the patient 401. For example, the accelerometer 216 of earbud 114b may provide acceleration data used by the tracking application 218 to determine the patient 401 is sleeping on their back. The tracking application 218 may further determine the angle at which the patient 401 is sleeping relative to the bed. Doing so may allow the tracking application 218 to provide a recommendation based on the determined sleep position and/or angle.

As stated, the tracking application 218 may further consider other wearables 110 when determining the sleep position of the patient 401. For example, based on an analysis of the data from the smartwatch 412 (e.g., raw data from the accelerometer 122 and/or orientation information determined by the smartwatch 412 based on the raw data from the accelerometer 122), the tracking application 218 may determine the orientation of the smartwatch 412 is consistent with the back sleeping position. For example, if, for the smartwatch 412, the sensor axis pointing upward shows a dominant gravitational pull (e.g., the Z-axis is approximately 9.8 m/s², and the X and Y axes are approximately zero), the tracking application 218 may determine the smartwatch 412 (and therefore the wrist of the patient 401) are facing upward, which is consistent with a back sleeping position (as a −9.8 m/s² Z-axis may indicate the smartwatch 412 (and/or wrist) is facing down towards to the bed, which may be consistent with a stomach sleeping position).

In addition and/or alternatively, the tracking application 218 may consider the chest monitor 410 when determining the sleep position of the patient 401. For example, based on an analysis of the data from the chest monitor 410 (e.g., raw data from the accelerometer 122 and/or orientation information determined by the chest monitor 410 based on the raw data from the accelerometer 122), the tracking application 218 may determine the orientation of the chest monitor 410 is consistent with the back sleeping position. For example, if, for the chest monitor 410, the sensor axis pointing upward shows a dominant gravitational pull (e.g., the Z-axis is approximately 9.8 m/s², and the X and Y axes are approximately zero), the tracking application 218 may determine the chest monitor 410 (and therefore the chest or torso of the patient 401) are facing upward, which is consistent with a back sleeping position (as a −9.8 m/s² Z-axis may indicate the chest monitor 410 (and/or torso) is facing down towards to the bed, which may be consistent with a stomach sleeping position).

Therefore, based on the data from the smartwatch 412, the tracking application 218 may determine the orientation of the wrist, and based on the data from the chest monitor 410, the tracking application 218 may determine the orientation of the torso. Therefore, the multimodal sleep position of the patient 401 may reflect the orientation and/or position of the head (based on the data from the earbud 114b and/or 114a), the torso, and the wrist. Based on any one or more of these positions and/or orientations, the tracking application 218 may generate a recommendation.

As stated, the tracking application 218 may monitor the movements of the patient 401, e.g., when the patient 401 gets into bed and/or gets out of bed. For example, over time, the tracking application 218 may determine that the patient has needed additional time to get out of bed in the morning (e.g., 10 seconds on average, up from a 5 second average). As such, the tracking application 218 may determine the existence of pathology such as Lewy body dementia in the patient 401. As such, the tracking application 218 may transmit an indication of the detected pathology, e.g., to the external device 104 of the patient's medical provider.

Similarly, the tracking application 218 may determine that the patient 401 falls at least once per week while getting out of bed. As such, the tracking application 218 may predict that the patient 401 may fall while getting out of bed. The tracking application 218 may determine when the patient 401 wakes up, and output a notification to use caution, ask for assistance, use a cane, etc., to prevent the predicted fall. The notification may be outputted via the earbuds 114a-114b, external devices 104 of the user, other wearables 110 of the user, etc. For example, the tracking application 218 of earbuds 114a-114b may transmit an instruction or other indication of the notification to the external devices 104 of the user and/or other wearables 110 of the user, e.g., via the network 112.

Further still, the tracking application 218 may track movements via the accelerometers 216 of the earbuds 114a-114b and/or other wearables 110 such as the chest monitor 410, smartwatch 412, ankle or leg-worn other wearables 110, etc. For example, if the other wearables 110 return data indicating the legs are moving, the tracking application 218 may analyze the data and determine the movements are consistent with restless leg syndrome. The tracking application 218 may then determine the patient 401 has restless leg syndrome, and transmit an indication of the pathology, e.g., to the external device 104 of the patient's medical provider.

As stated, the tracking application 218 may consider additional factors when generating recommendations for improving health. For example, as shown, a soundwave 406 and/or a soundwave 408 may be detected by the microphone arrays 208 of the earbud 114a and/or 114b. The location the soundwaves 406, 408 originated may be determined by the tracking application 218 as described herein.

For example, the tracking application 218 may generally determine the soundwave 406 originated at a distance from the head of the patient 401. Similarly, the tracking application 218 may analyze the soundwave 406 and determine that the soundwave 406 is associated with sounds made by the RPT device 106. Therefore, the tracking application 218 may determine the patient 401 is using the RPT device 106. Based on the determination that the patient 401 is using the RPT device 106, the tracking application 218 may determine a pathology of the patient, e.g., that the patient 401 has OSA, positional sleep apnea (POSA), central sleep apnea (CSA), etc. In some embodiments, the tracking application 218 may generate a recommendation based on the sleep position of the patient 401, the use of the RPT device 106, and/or the determined pathology.

Furthermore, the tracking application 218 may generally determine the soundwave 408 originated close to the face of the person. Similarly, the tracking application 218 may analyze the soundwave 408 and determine that the soundwave 408 is associated with an airflow obstruction in the air circuit 404. The tracking application 218 may determine that the airflow obstruction is caused by the sleep position of the patient 401, e.g., the face and/or other parts of the body may be impinging the air circuit 404. Because the obstruction in the air circuit 404 may result in a reduction (and/or cessation) of the therapy delivered to the patient 401, the tracking application 218 may generate a recommendation, e.g., generate a noise via the speakers 206 of the earbuds 114a, earbud 114b, to wake the patient 401 such that the sleep position is changed and blockage in air circuit 404 is cleared. Embodiments are not limited in these contexts.

In some embodiments, a system 504 may be provided for measuring physiological parameters, e.g., characteristics of a patient while they are awake or asleep. The system may be configured to provide a patient 501 with ongoing monitoring of their waking physiological parameters, e.g., an “awake state” to determine, e.g., quantify, whether their sleep quality improves as a result of respiratory therapy, e.g., PAP therapy. In this regard, the system 504 may be considered a patient tracker 504 configured to measure a patient's physiological state during the day, rather than at night.

Returning to FIG. 5A, the system 504 may be implemented in the form of a wearable device, such as the “earbud” type wearable device shown wearable with respect to a person's ear canal, ear lobe or behind the person's ear. In this regard, the patient tracker 504 may be considered a patient tracking device 504 (also referred to as device 504). Therefore, in at least one embodiment, the device 504 is the earbud 114a and/or earbud 114b.

The patient tracking device 504 may be configured to measure daytime activities and physiological characteristics, e.g., conditions, of the patient. For example, the system 504 may be configured to measure activities such as: a distance travelled by the patient; a number of steps (or paces) walked, run, climbed, etc.; a type, duration, intensity, etc., of physical activity; time spent standing.

The daytime activities set forth above may influence the physiological characteristics of the patient. For example, a patient travelling a distance may have an elevated heart rate, increased breath rate, increased fatigue, etc. The physiological characteristics that can be measured by the system include: a respiration rate, variability of respiration, etc.; a heart rate, variability of heart rate, etc.; a magnitude of calories burned; blood oxygen saturation; electrodermal activity (e.g., skin conductance or galvanic skin response); electrical activity of the brain; eye blinks; or any combination thereof.

The device 504 may be used by itself, or independently of, e.g., without a the RPT device 106. In particular, the device is shown without a patient interface 601. In this form, the patient may, for example, wear the device by itself during daytime activities such as walking, running, etc.

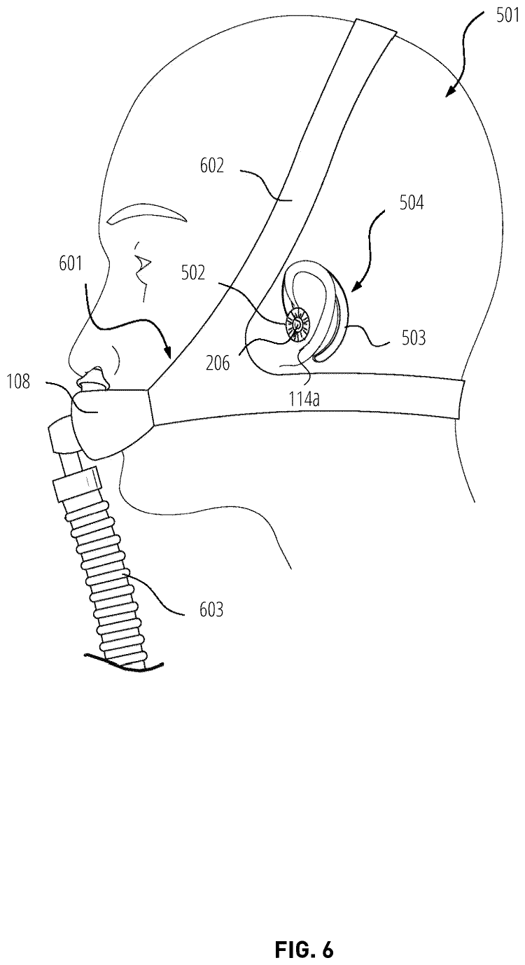

By comparison, and as shown in FIG. 6, the device 504 may also be used together with an RPT device such as RPT device 106. In the form shown, the device is worn together with a patient interface 601 (as part of the RPT device 106). The patient may wear the device in this way, e.g., with the patient interface 601, while they sleep for recording data while also receiving respiratory therapy.

As shown in FIG. 5A, FIG. 5B, and FIG. 6, the device 504 comprises a body 502 for housing the control system, memory device, sensors, batteries (rechargeable or replaceable), etc. An ear hook 503 is provided for locating, e.g., attaching, mounting, etc., the device 504 about the patient's ear. In particular, the ear hook is configured, e.g., shaped, to locate and removably secure behind the patient's external ear (e.g., auricle/pinna).

The body 502 is configured to locate within the patient's ear for transmitting audio (e.g., sound) into the ear for the patient to hear. At least a portion of the body 502 may be configured to releasably secure within at least a portion of the patient's external auditory canal. In this regard, the body of the device may be shaped similar to a traditional earbud used for transmitting audio into a patient's ear.

The patient tracker 504 may be configured to receive the physiological data about the patient from the one or more sensors (e.g., sensors 204, not pictured for clarity). In some forms, the patient tracker may also be configured to receive environmental data from the one or more sensors.

The environmental data may relate to environmental conditions surrounding the patient (e.g., the environmental data being related to the patient), such as temperature, humidity, etc. In either form of data, e.g., physiological or environmental, the data may be stored in the memory device and analyzed by the processor(s) of the control system.

Advantageously, measuring and recording data relating to the environmental conditions surrounding the patient may allow the device 504 to accommodate for environmental conditions that influence the physiological conditions of the patient. For example, if the humidity and temperature of air surrounding the patient is high, the patient may fatigue more rapidly when e.g., walking, than in colder, less humid conditions. In effect, environmental conditions (such as high humidity and temperature) may inadvertently indicate the patient is fatigued as a result of e.g., a lack of sleep. Hence, allowing the device 504 to accommodate for such environmental conditions means that indications of the patient's sleep quality can be more accurately presented to the patient.

For example, an optical sensor using red, infrared, and/or green, could be used to calculate a photoplethsmogram. Subsequently, parameters such as pulse rate (PR), pulse rate variability (PRV), SpO₂ can be determined. If respective sensors are placed on a periphery of the user, e.g., at their skin, the peripheral arterial tone may also be measured.

Generally, the types of sensors 204 utilized in the patient tracker 504 may vary according to the physiological and/or environmental data being generated. For example, when the patient tracker 504 is integrated into an item of clothing, it may comprise the electromyography (EMG) sensor for detecting electrical signals generated by muscles. Alternatively, the EMG sensor may not be utilized when the patient tracker is integrated into a ring. In any case, each of the one or more sensors may be configured to output sensor data that is received and stored in the memory device of the patient tracker 504.

In some forms of the device 504, one or more of the sensors set forth above may be configured to contact the patient's skin. In this regard, the sensors may be located on an externally facing surface of the body 502 or the ear hook 503, so as to be in contact with the patient's skin when in use. This allows, e.g., the galvanic skin response (GSR) sensor, to measure changes in sweat gland activity on the skin. In another example, the one or more sensors, e.g., optical sensor, may be located on the ear hook so as to contact an area of skin between the patient's auricle/pinna and hairline.

Other forms of the sensors may be configured for mounting internally to the body 502 and ear hook 503, such as the motion sensor (e.g., accelerometers 216). In this case, for example, the motion sensor may be integrated within the body of the device and configured to measure a patient's head movement.

As set forth above, the one or more sensors of the patient tracker 504 can be configured to determine an awake state of the patient. The patient tracker utilizes the physiological and environmental data generated from the sensors to determine how “awake,” e.g., alert, the patient is for a duration of non-sleep, e.g., during the daytime. For example, the device 504 may be configured to measure a patient's heart rate and EEG during the daytime. Based on the physiological data generated from variations in the heart rate and EEG measurements, the system 504 may indicate how awake the patient is, e.g., if the patient is lethargic and has an unfocussed attention during the daytime.

In order to determine an awake state, including stages of a sleep (such as NREM (N1, N2, N3/SWS) or REM), data may be fed into an artificial Intelligence (AI) or Machine Learning (ML) model such as the one or more of the models 220. This model 220 may be trained on the IMU and PPG signals, or pre-processed parameters of those.

Breathing and/or respiration signal related parameters can include: variability of breathing rate throughout the day and/or night (the variability being characteristic of the person)—this can be inter-breath or over longer timescales—e.g., 30, 60, 90 sec or much longer periods; the stability over time (related to the variability); the standard deviation of breathing rate; the depth of respiration (shallow, deep etc.), and relative amplitude of adjacent breaths; the mean or average value of the breathing rate; the trimmed mean (e.g., at 10%) to reject outliers; wake or Asleep (e.g., the detected sleep stage of the person); surges (sudden accelerations or decelerations) in breathing rate seen during quiet periods and during REM sleep; median (50th percentile); interquartile range (25th-75th percentile); 5th-95th percentile; 10th-90th percentile; shape of histogram; skewness; kurtosis; peak frequency over time; ratio of second and third harmonics of peak frequency; percentage of valid data (Valid Physiologically Plausible Data); autocorrelation of the individual signals; characteristic patterns in the spectrogram; wake or asleep; relative percentage of REM and deep sleep.

Cardiac signals can be processed to produce features such as: heart rate variability HRV (inter beat (e.g., as derived from the Ballistocardiogram) and over longer defined moving windows—e.g., 30, 60, 90 sec); variability over time (interbeat/breath variability); mean; trimmed mean (10%); standard deviation; median (50th percentile); interquartile range (25th-75th percentile); 5th-95th percentile; 10th-90th percentile; shape of histogram; skewness; kurtosis; stability over time; peak frequency over time; ratio of second and third harmonics of peak frequency; percentage of valid data (Valid Physiologically Plausible Data), wake or asleep; autocorrelation of the individual signals; characteristic patterns in the spectrogram.

Cardiorespiratory signals can be formed, such as: magnitude square cross spectral density (in a moving window); cross coherence; respiratory sinus arrhythmia peak; low frequency (LF)/high frequency (HF) ratio to indicate autonomic nervous system parasympathetic/sympathetic balance (LF is often defined as around 0.04-0.15 Hz, whereas HF is around 0.15-0.4 Hz); the cross correlation; cross coherence (or cross spectral density) of the heart and breathing signal estimates; non-linear estimates such as entropy measures; the characteristic movement patterns over longer time scales, e.g., the statistical behavior observed in the signals; patterns of movement during detection of and comparison of these heart and breathing signals (e.g., during sleep, some people may have more restful and some more restless sleep).

Based on the determination of a patient's awake state, the patient tracker 504 may provide the patient with an indication of how effective their respiratory therapy, e.g., PAP therapy, is at improving their sleep quality. For example, in the case where physiological data indicates the patient is lethargic and unfocussed, such an indication may be correlated with a low efficacy of the patient's PAP therapy.

Conversely, in the case where physiological data indicates the patient has improved capacity for daytime activities, e.g., a lower resting heart rate, etc., such an indication may be correlated with a high efficacy of the patient's PAP therapy. As set forth in more detail later, the patient tracker may be configured to alert the patient of such indications, e.g., notifications that e.g., a morning run, positively impacted their sleep.

The patient tracker 504 may be configured to measure an efficacy of respiratory therapy by recording a baseline measure of “off therapy” physiological data and comparing this to an “on therapy” measure of physiological data. According to the changes detected in the measured data, the patient tracker may advise the patient of either improvements to their sleep performance, or deteriorations to their sleep performance.

In a variation, the patient may be advised of improvements that occur in their ability to undertake daytime activities, such as capacity for exercise, that are a result of their corresponding improvements to their sleep performance. Conversely, the patient tracker can be configured to notify the patient of a deteriorated capacity to perform daytime activities as a result of a corresponding deterioration in their sleep performance. Advantageously, notifying a patient of said changes to either their sleep performance or capacity for daytime activities can allow a patient to understand an impact of their respiratory therapy.

As part of providing the patient with an indication of how effective their respiratory therapy is, the patient tracker 504 may also be configured to record, e.g., “timestamp” events associated with the patient's sleep periods. For example, the various sensors of the patient tracker 504 may be configured to record a time that the patient wakes after a period of sleep, times when the patient wakes during a period of sleep (e.g., a rate of sleep disturbances), a time that the patient exits the bed, a time that the patient enter the bed, etc. These events may be utilized, e.g., analyzed, together with other sensor data gathered about the patient, to determine how awake the patient may be as a result of their e.g., PAP therapy.

Advantageously, data relating to e.g., when a patient wakes, may be longitudinally recorded so as to determine sleeping patterns of the patient. This information may be processed and utilized to inform the patient of e.g., whether they are ready for sleep; whether they are sleeping well; whether they should expect to feel tired during their waking hours, etc. Ultimately, the patient tracker 504 may provide the patient with an indication of how effective their respiratory therapy has been.

Set forth below are some further examples of sensors such as sensors 204 that may be used with the patient device 504, and their application for use with the patient device 504.

In some forms of the patient tracker 504 where the motion sensor is utilized (as set forth previously), the motion sensor may generate data relating to specific movements of the patient, such as exercise (e.g., running), or other body (e.g., limb) movements. These movements may be utilized to determine the patient's awake state. For example, a patient's limb movements may be analyzed and determined as being slow relative to a standard measurement of the patient's normal movements.

While the motion sensor is described in broad terms, the motion sensor may be specifically one or more inertial sensors, such as accelerometers, gyroscopes, and magnetometers. These types of motion sensors may be selected, e.g., utilized according to their optimal use-case.

In some forms of the motion sensors, the motion sensors may be configured to detect motion or acceleration associated with arterial pulses, such as pulses in or around the face of the patient and in particular, those proximal to the patient tracking device 504, e.g., the body 502. The motion sensors in this form may be configured to detect features of the pulse shape, speed, amplitude, or volume that may be analyzed to indicate qualities of a patient's awake state.

In other forms, an EEG sensor may also be provided in the patient tracker 504 for measuring physiological data relating to the patient's brain. The EEG sensor may include one or more dry electrodes positioned on or around the scalp of the patient. In this form, the EEG may locate within, or extend from, a portion of the ear hook 503 or body 502. For this reason, the EEG sensor is optimally utilized when the patient tracker is implemented as an earpiece, as shown in FIG. 5A, FIG. 5B, and FIG. 6, such that the external surfaces of the body 502 and car hook 503 may be in contact with the patient's scalp.

Depending on the placement of the EEG sensors, it may be possible to detect EEG slowing during the daytime (such as a higher ratio of delta and theta frequencies to alpha and beta frequencies) and relate this daytime slowing to greater daytime sleepiness. Thus, it may be possible to avoid asking a patient if they have daytime sleepiness, but rather, derive it from EEG slowing vs. a baseline and relate this to a reduced movement of the patient (detected from e.g., an accelerometer such as accelerometer 216).

In forms where a PPG sensor is provided to measure, e.g., a heart rate, the patient tracker is optimally configured to contact the patient's skin. In this form, the patient tracker may be integrated into a piece of clothing to optimally generate data relating to e.g., a heart rate pattern, a heart rate variability, a cardiac cycle, respiration rate, estimated blood pressure, or any combination thereof.

When the patient tracker is integrated into an earbud and/or earpiece as shown in FIG. 5A, FIG. 5B, and FIG. 6, a speaker 206 may be provided for outputting (e.g., generating) audio. The audio, e.g., generated sounds, are configured to be projected into the patient's ear so as to be heard by the patient. For example, the patient tracker 504 may be configured as a type of carphone to play music for a patient to listen to during the day. In another example, the patient tracker 504 may be configured to sound an alarm for waking the patient from sleep, reminding them of an event (e.g., a calendar event). In yet a further example, the device 504 may assist in relaxation of the patient prior to sleep by playing controlled breathing audio cues. In yet a further example again, the device 504 may also provide hearing assistance, whereby the device may be coupled with a smartphone to generate audio, amplify audio, etc.

In some implementations, the speaker 206 may be used together with, or substituted by, a bone conduction speaker. In this form, the bone conduction speaker is not configured to generate audio for the patient to hear via their cars, rather, the speaker generates vibrations that are configured to penetrate the patient's temporal bones. In variations, an audio and bone conduction speaker may be configured for use together.

In some further implementations, the speaker 206 may be a noise cancelling speaker for assisting in reduction of background noises. Advantageously, this may be used prior to sleep, for reducing background noises that may otherwise hinder sleep.

In either form of the speaker, e.g., as a speaker, bone conduction speaker, noise cancelling speaker, etc., the patient tracking device 504 may be coupled (e.g., wired or wirelessly) to a computing device such as external device 104, e.g., a mobile phone, for playing music or otherwise generating the sounds for the patient to hear. In the case of a wireless connection, the patient tracking device 504 may be configured to communicate through various communication protocols, such as, Wi-Fi, Bluetooth, etc. The patient tracking device may thereby include an antenna, a receiver, a transmitter, a transceiver, or any combination thereof for communicating with wirelessly with a computing device.

The external device 104 may be configured to operate, e.g., run software configured to communicate with the patient tracking device 504. In forms where the external device 104 is a mobile phone or tablet, the software may be configured as a mobile application allowing the patient to control operation of the patient tracking device via the mobile device.

The external device 104 may be used as a way to display information about the patient's awake state or other data in the user profiles 224. In other forms, the computing device may also be configured to process (via one or more processors) data generated from the patient tracking device 504. In further forms, the external device 104 may be configured to receive input from the patient for controlling operation of the patient tracking device. As set forth above, the input of the patient may relate to the patient configuring the patient tracking device 504 to send diary alarms, or in other cases, to select music to listen to (via the speakers).

In some forms of the patient tracking device, the patient may input information into the computing device, e.g., via the software, for determining, at least in part, the awake state of the patient. That is, the patient may self-report information that may not be sensed, per se, but be provided by the patient to be considered together with physiological and/or environmental data generated by the sensors. The combination of self-reported data and sensed data may be analyzed to determine a patient's awake state.

The self-reported information input by the patient may include demographic information, biometric information, falls, therapy device use, medical information such as medications, etc., diet(s), adverse health events, subjective stress level of the patient, subjective fatigue level of the patient, subjective health status of the patient, a recent life event experienced by the patient, or any combination thereof. In the case of the medical information, the patient may provide information relating to one or more medical conditions, medication usage, etc.

Referring to FIG. 6, the patient tracking device 504 (e.g., earbud 114a or 114b) may be configured for use with respiratory therapy, e.g., an RPT device such as RPT device 106. The RPT device may include the patient interface 601, a conduit 603, a mask 108 and a positioning and stabilizing structure 602. It is noted that although a particular mask is shown in FIG. 6, other types of masks may be utilized, such as a full-face mask, nasal mask, oro-nasal mask, etc.

As set forth above, the patient tracker 504 may provide the patient with ongoing monitoring of their “awake” state and provide feedback to the patient regarding any differences detected between “on” and “off” therapy. In other words, the patient tracker 504 may indicate changes in the patient's sleep performance after they begin respiratory therapy and, in effect, indicate to the patient how effective their use of respiratory therapy has been.

The patient tracker 504 may be configured to correlate changes in a patient's daytime activities with their adherence and/or compliance to e.g., CPAP therapy. For example, in patients having symptoms such as chronic fatigue, daytime sleepiness, cognitive impairment, etc., the patient tracker (as set forth previously) may be configured to monitor for improvements in such symptoms. The patient tracker may be configured to determine correlations between these improvements, e.g., changes, and the patient's adherence and/or compliance to CPAP therapy. These correlations may be reported, e.g., communicated as feedback to the patient, so that the patient is aware of the positive impact their adherence and/or compliance to CPAP therapy has on their capacity for performing daytime activities.

The patient tracker 504 may be configured to interrelate a specific respiratory therapy, e.g., CPAP and a deterioration of healthy behaviors or an improvement of healthy behaviors. That is, the patient tracker 504 may also be configured to monitor and report to a patient their unhealthy behaviors which may occur as a result of sleep related breathing disorders.

For example, patients having sleep apnea for an extended period of time prior to diagnosis may develop unhealthy behaviors, such as lack of exercise, bad sleep habits, etc., which may persist even after commencing respiratory therapies, e.g., CPAP. The sensor(s) and self-reported information input into the patient tracker 504 may be used to monitor and report to the patient such behaviors. Reporting these behaviors as feedback to the patient may assist the patient to change, e.g., re-train such behaviors. Advantageously, re-training the patient to remove said unhealthy behaviors can positively impact their respiratory therapy, in addition to reducing the patient's risk of comorbidities.

In this regard, the patient tracker 504 can provide the patient with a complete treatment for their sleep related breathing disorder(s). That is, in addition to opening the patient's airways via, e.g., PAP therapy, the patient tracker can identify and treat unhealthy behaviors that are symptomatic of the sleep related breathing disorder. Advantageously, this can motivate a patient to be more adherent and/or compliant to respiratory therapy.

In some forms, the patient tracker 504 may be configured to provide the patient with detailed correlations of their improved daytime activities and corresponding compliance to respiratory therapy. For example, the patient tracker 504 may be configured to record use of therapy devices and correlate a patient's use of CPAP therapy during a sleep period, with the patient being able to run a larger distance the following day, or the patient having a lower resting heart rate, etc.

The patient tracker 504, as set forth above, can be configured to improve a patient's adherence and/or compliance by behavioral intervention. That is, the patient tracker 504 can be configured to allow a patient to break, e.g., intervene, particular habits that are associated with their sleep related breathing disorder(s).

In some forms, a patient's compliance and/or adherence to a respiratory therapy may also be detected by measurements taken by the one or more sensors of the patient tracker 504. For example, the patient tracker may include an EEG configured to measure daytime markers of a patient's increased alertness. Such markers may be compared against normal measures of the patients' alertness, such that an indication of the patients' improved alertness can be determined. This can indicate an improved efficacy of the respiratory therapy, and in turn, indicate the patients' adherence and/or compliance to therapy. Advantageously, use of the sensors to automatically detect efficacy and therapy adherence and/or compliance means that the patient may not be required to monitor their perceived daytime sleepiness, e.g., lethargy or reduced alertness to determine an efficacy of their respiratory therapy.

Furthermore, utilizing the EEG for compliance indications may also allow for a detection of impaired cognitive function. That is, detection of a patient's alertness may be used as a proxy for an assessment of their cognitive function.

In some forms, the patient tracking device 504 may be coupled with the RPT device 106 to monitor the patient's sleep stage during periods of sleep. In this form, the sensors of the patient tracking device 504 may be used together with the sensors of the RPT device 106 (e.g., optionally located in the patient interface 601, flow generator, or other component of the RPT device 106), for detecting e.g., states of a sleep cycle. In some embodiments, the data collected may be used to determine a sleep position of the patient. In some forms, the data collected may be used to inform the patient of how effective their respiratory therapy has been, and in other forms the data may be additionally or alternatively used to adjust the delivery of respiratory therapy, e.g., pressure, flow rate, etc. Therefore, the earbuds 114a, 114b, and/or the tracking application 218, may instruct the RPT device 106 to adjust pressure, flow rate, etc., based on a sleep position detected by the tracking application 218.

As stated, one or more microphone arrays 208 may also be provided to the patient tracking device 504 to measure a patient's breathing during sleep. In this form, a microphone array 208 may be located proximal to the patient's mouth and/or nose, and so accurately record breathing sounds, e.g., in the user profiles 224. A detection of abnormal breathing may be indicative of a sleep apnea, whereby the patient tracking device (e.g., earbuds 114a-114b) may be used together with the RPT device 106 to adjust therapy, e.g., pressure, flow, etc., for stimulating a change in the patient's breathing.

In some further forms, one or more motion sensors 204 described previously may be utilized during a patient's sleeping period to detect movements of the patient. For example, a number of movements during a sleep period may be detected, and used to provide an indication of e.g., a disturbed sleep. In some forms, the data collected from the motion sensor may be fused, e.g., coupled, combined, integrated, etc., with flow data collected from the RPT device 106. This combination of data may be used to improve sleep and/or wake classification, e.g., determination of a patient's awake state.

In other forms, the patient tracker 504 may be configured to monitor the patient's sleep stage without being coupled to the RPT device 106. In this form, the patient tracker 504 may be configured to detect and record physiological and/or environmental data as it would when coupled with the RPT device 106. However, rather than adjust operation of the RPT device, the patient tracker 504 in this form would utilize the data recorded to inform the patient of their sleep performance, e.g., apnea events, etc.

In some further forms where the patient tracking device 504 is used without being coupled to the RPT device 106, the data collected during a sleeping period may be implemented as a change to respiratory therapy at a later date. That is, the data collected when the patient is not wearing the patient interface 601 may be used to adjust therapy the next time the patient wears the patient interface 601.

In some further forms, the patient tracking device 504 may be configured to intermittently couple with the RPT device 106 so as to communicate with the RPT device. The patient tracking device 504 in this form may be configured to operate both together with the RPT device 106, and independently of the RPT device 106. That is, when the patient is near the RPT device 106, the patient tracker 504 may be able to connect (e.g., wirelessly) with the RPT device 106. When the patient is away from the RPT device 106, e.g., walking outside, the patient tracker may be able to operate independently of the RPT device 106.

For example, the patient tracker 504 operating independently may be able to temporarily record and store data from the sensors for later communicating said data to the RPT device 106 when the patient tracker 504 is proximal to the RPT device 106.

In this form, the patient tracking device 504 may be worn together with the patient interface 601 in some instances, e.g., during sleep, and in other instances the patient tracking device may not be worn with the patient interface 601, e.g., when a patient leaves their home. In some cases, missing data e.g., data which is not collected by either the RPT device 106 or patient tracking device 504, may be collected from an alternative data source, such as a wrist worn accelerometer or heartrate sensor. For example, the device 504 may be coupled with an external device 104 such as a smart watch, or a smart hub for collecting data that may not be captured by the device 504 or the RPT device 106.

In some forms, the external device 104, e.g., mobile device, as set forth previously may be configured to connect with the patient tracker 504 when the patient tracker 504 is not coupled with the RPT device 106. In this regard, the device 504 may be configured to log and process data within its memory, without requiring a wireless connection for a period of time.

In some forms, the patient tracker 504 may be utilized for detecting and diagnosing a patient with an un-treated sleep related breathing disorder. The patient tracker 504 used in this form may allow a patient to determine whether they require respiratory therapy e.g., PAP therapy, positional therapy, insomnia treatment, etc. In this form, the sensors (as set forth previously) may be configured to register (e.g., detect) a sleep event that is indicative of a sleep related breathing disorder.

In forms whereby the patient tracker 504 is configured for detecting and diagnosing a patient with a sleep related breathing disorder, the patient tracker 504 may be utilized to monitor a patient's daytime activities to determine indications of sleep related breathing disorders. For example, a patient may develop unhealthy behaviors, such as lack of exercise, bad sleep habits, etc., that may be detected and utilized as an indicator of insomnia, etc.

In some embodiments, the patient tracker 504 may be utilized for detecting a patient with an under-treated sleep related breathing disorder. That is, a patient having already been diagnosed with a sleep related breathing disorder, but is not receiving effective therapy. In this case, the patient tracker 504 may be configured to monitor e.g., heart rate variability for indicating whether the patient is under-treated. In response, the patient tracker 504 may be configured to provide the patient with an indication of how to adjust therapy during the night, or alternatively, the patient tracker may be configured to automatically adjust a respiratory therapy device (as set forth previously) to appropriately treat the under-treated disorder.

In some forms, the patient tracker 504 can also be configured for detecting and monitoring for comorbidities of sleep apnea. For example, the sensor(s) set forth above may be configured for detecting diabetes, heart failure, stroke, and obesity.

FIG. 7 is a schematic 701 illustrating an example of using earbuds to detect reaction times, movements, tasks, sleep positions, therapy devices, airway obstructions, and/or other pathologies, in accordance with one embodiment. FIG. 7, which is not to scale, depicts the ear canals 703, 704 of a human head 702. Furthermore, two microphones (or microphone arrays) of an earbud (not pictured) are depicted in the ear canal of the person. For example, microphones 706a and 706b (denoted by M1 and M2, respectively) may be included in a first earbud (not pictured) located at or near ear canal 703. Similarly microphones 706c and 706d (denoted by M3, and M4, respectively) may be included a second earbud (not pictured) located at or near ear canal 704. The microphones 706a-706d are representative of one or microphones and/or microphone arrays. For example, each microphone microphones 706a-706d may be representative of a respective microphone array 208. In some embodiments, to determine the location of a sound emitted from a sound source 705, any two or more of the microphones 706a-706d may define a microphone array. Embodiments are not limited in these contexts.

As stated, the distances between any two of microphones 706a-706d may be known or otherwise determined. As shown, as one or more soundwaves 707 generated at sound source 705 moves through space, portions of the soundwaves 707 may enter each ear canal 703, 704. Therefore, portions of the soundwaves 707 may be detected by the microphones 706a-706d at different times. For example, microphone 706a may detect soundwaves 707 prior to the time microphone 706b detects soundwaves 707. Similarly, microphone 706d may detect soundwaves 707 prior to microphone 706c.

Therefore, the phase relationship between the soundwaves 707 detected at each microphone 706a-706d may be used to determine the location of the sound source 705. For example, the tracking application 218 of an earbud may determine the phase shift of soundwave 707 between microphone 706a and microphone 706b, while the tracking application 218 of the other earbud in the pair may determine the phase shift of soundwaves 707 between microphone 706c and microphone 706d. As another example, one earbud may determine the phase shift of soundwaves 707 between microphone 706a and microphone 706d. Embodiments are not limited in these contexts, as the phase shift may be determined between any two or more of the microphones 706a-706d. The tracking application 218 may then compare or otherwise use the phase shifts to triangulate the location of the sound source 705, e.g., using triangulation, trilateration, time difference of arrival (TDOA), etc. For example, by converting phase shifts to time differences, the TDOA between multiple (e.g., two or more of) microphones 706a-706d may be computed to determine the location of the sound source 705.

The tracking application 218 may determine that the soundwaves 707 are associated with a position of the patient based at least in part on the determined location of the sound source 705. For example, by determining the sound source 705 is centrally located a few centimeters away from the earbuds 114a, 114b (e.g., in the patient's throat), the tracking application 218 may determine the patient is the sound source 705. Based on an analysis of the soundwaves 707, the tracking application 218 may determine the sound is associated with snoring, sleep apnea events, etc., e.g., a pathology. Based on detecting the pathology and location thereof, the tracking application 218 may generate a recommendation. For example, based on a determination that the person is sleeping in a supine position, the determined location of the soundwaves 707, and the detection of the pathology, the tracking application 218 may generate a recommendation, e.g., to cause the patient to change sleep positions, modify parameters of the RPT device 106, etc.

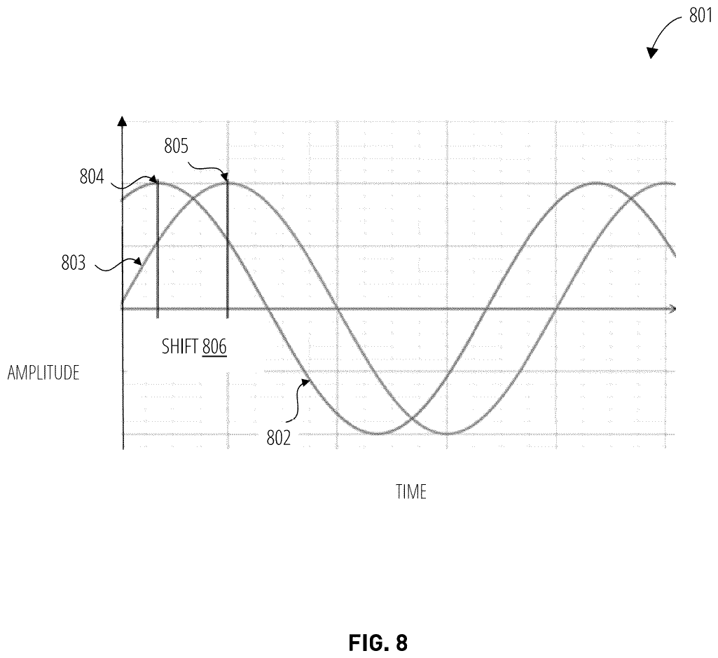

FIG. 8 is an example graph 801 depicting two waveforms of a sound, where the x-axis corresponds to time and the y-axis corresponds to amplitude. As shown, waveforms 802 and 803 are depicted in the graph 801. In one example, the waveform 802 may be detected by microphone 706a, while waveform 803 may be detected by microphone 706b. Although the waveforms 802 and 803 appear similar, they are shifted due to the different times the sound is detected by a respective microphone, which is based on the distance between the microphones and the speed of sound. Therefore, the phase shift 806 of waveform 802, 803 may be determined according to any suitable technique. For example, determining the phase shift at two points 804, 805 may be based on cross-correlation which measures the similarity between waveforms 802, 803 as a function of the time-lag applied to one of them. For example, the cross-correlation may be computed using a Fast Fourier Transform (FFT). As another example, the time difference between points 804, 805 may be computed. The cross-correlation and/or time difference may be a time shift. The time shift may be used to compute phase shift of the waveforms 802, 803.

As another example, the tracking application 218 may compute phase shift 806 by comparing the phases of waveforms 802, 803 in the frequency domain. For example, the tracking application 218 may compute the Fourier Transform of both waveforms 802, 803 to determine their frequency components. The tracking application 218 may identify the phases of the frequency components and compute the phase shift based on computing the difference of the identified phases of the frequency components. More generally, once the time shift and/or phase shifts are determined, the location a sound originated may be determined. For example, using the phase and/or time shift between microphones 706a, 706b, and 706c may be used to determine TDOA at each microphone, which may then be used to determine the location a sound originated. Embodiments are not limited in these contexts.

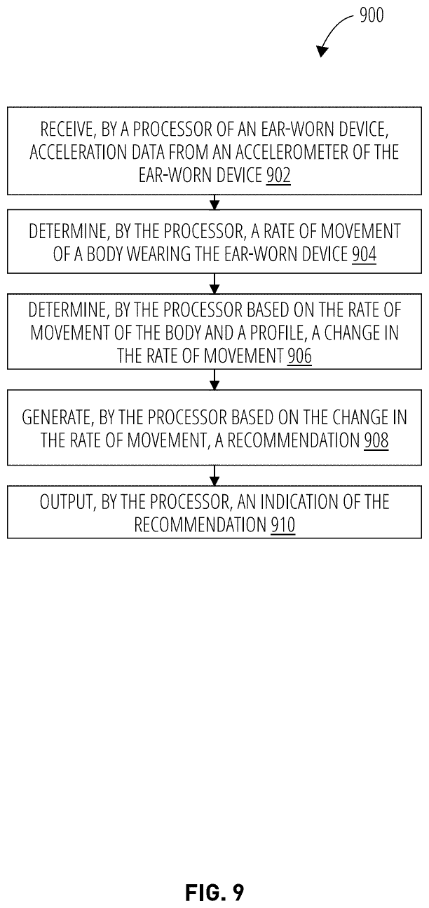

FIG. 9 illustrates an example logic flow 900 for tracking reaction time using ear-worn devices, according to one embodiment. Although the example logic flow 900 depicts a particular sequence of operations, the sequence may be altered without departing from the scope of the present disclosure. For example, some of the operations depicted may be performed in parallel or in a different sequence that does not materially affect the function of the logic flow 900. In other examples, different components of an example device or system that implements the logic flow 900 may perform functions at substantially the same time or in a specific sequence.

According to some examples, the logic flow 900 includes receiving, by a processor of an ear-worn device, acceleration data from an accelerometer of the ear-worn device at block 902. For example, the processor 116e of earbud 114a may receive acceleration data from an accelerometer 216 of the earbud 114a.

According to some examples, the logic flow 900 includes determining, by the processor, a rate of movement of a body wearing the ear-worn device at block 904. For example, the processor 116e may determine a rate of movement of a body wearing the earbud 114a. For example, the processor 116e may determine the person is standing up and required 4 seconds to stand up.

According to some examples, the logic flow 900 includes determining, by the processor based on the rate of movement of the body and a profile, a change in the rate of movement at block 906. For example, the processor 116e may determine, based on the rate of movement of the body and the user profile 224, a change in the rate of movement. For example, by tracking prior instances of when the person stands, the user profile 224 may reflect the average amount of time required by the person to stand. If the time detected at block 904 exceeds the average amount of time, the processor 116e may detect the change. Similarly, by computing averages over time intervals, the processor 116e may detect the change (e.g., if the time to stand over consecutive one month periods is four seconds, fix seconds, six seconds, etc.) over these intervals. Embodiments are not limited in these contexts.

According to some examples, the logic flow 900 includes generating, by the processor based on the change in the rate of movement, a recommendation at block 908. For example, the processor 116e may generate, based on the change in the rate of movement, a recommendation. For example, the increased time required for the person to stand may be a symptom of a pathology. Therefore, the recommendation may include a notification to the person, e.g., to exercise caution while standing, recommend using a cane, transmitting an indication of the pathology to the external device 104 of the person's medical provider, etc.

According to some examples, the logic flow 900 includes outputting, by the processor, an indication of the recommendation at block 910. For example, the processor 116e may output an indication of the recommendation.

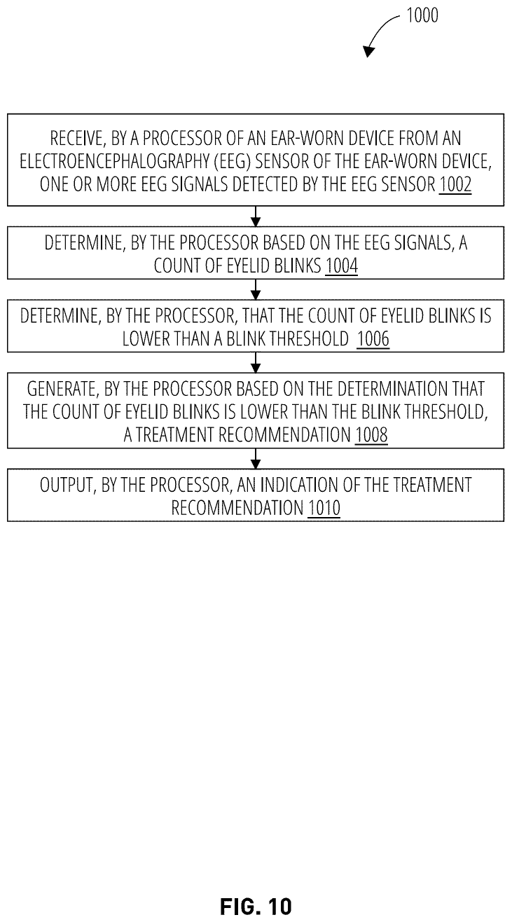

FIG. 10 illustrates an example logic flow 1000 for tracking reaction time using car-worn devices, according to one embodiment. Although the example logic flow 1000 depicts a particular sequence of operations, the sequence may be altered without departing from the scope of the present disclosure. For example, some of the operations depicted may be performed in parallel or in a different sequence that does not materially affect the function of the logic flow 1000. In other examples, different components of an example device or system that implements the logic flow 1000 may perform functions at substantially the same time or in a specific sequence.

According to some examples, the logic flow 1000 includes receiving, by a processor of an ear-worn device from an electroencephalography (EEG) sensor of the ear-worn device, one or more EEG signals detected by the EEG sensor at block 1002. For example, the processor 116e of earbud 114a may receive, from an electroencephalography (EEG) sensor of the earbud 114a, such as electrodes 214, one or more EEG signals detected by the EEG sensor.

According to some examples, the logic flow 1000 includes determining, by the processor based on the EEG signals, a count of eyelid blinks at block 1004. For example, the processor 116e may determine, based on the EEG signals, a count of eyelid blinks. For example, the processor 116e may detect blink artifacts in the EEG signals.

According to some examples, the logic flow 1000 includes determining, by the processor, that the count of eyelid blinks is lower than a blink threshold at block 1006. For example, the processor 116e may determine that the count of eyelid blinks is lower than a blink threshold in the user profile 224 for the user.

According to some examples, the logic flow 1000 includes generating, by the processor based on the determination that the count of eyelid blinks is lower than the blink threshold, a treatment recommendation at block 1008. For example, the processor 116e may generate, based on the determination that the count of eyelid blinks is lower than the blink threshold, a treatment recommendation. For example, the processor 116e may determine to wake the person, engage autonomous driving features if the person is driving a car, etc. In some embodiments, the processor 116e may consider a level of fatigue. The processor 116e may determine the level of fatigue by computing a score based on the eyelid blinks, the threshold, one or more characteristics of brain waves detected by the EEG sensors, etc.

According to some examples, the logic flow 1000 includes outputting, by the processor, an indication of the treatment recommendation at block 1010. For example, the processor 116e may output an indication of the treatment recommendation, e.g., by outputting sounds via the speakers 206, emitting vibrations via the haptic feedback modules 212, transmitting instructions to the external device 104 of the vehicle, etc.

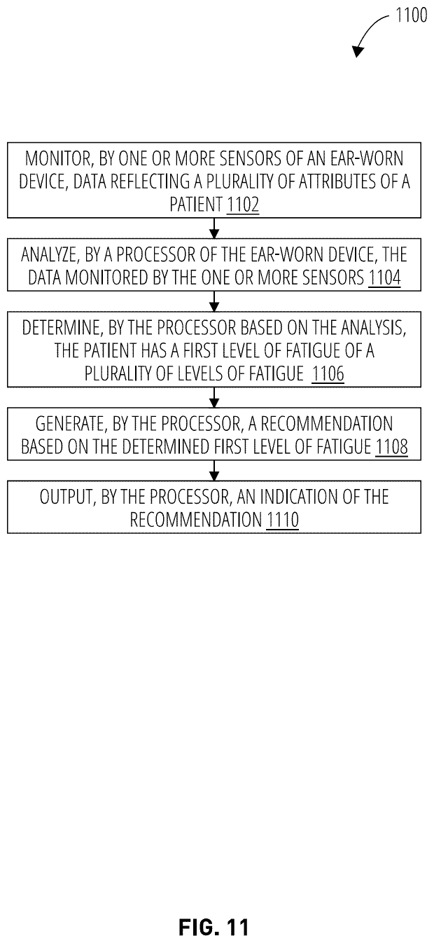

FIG. 11 illustrates an example logic flow 1100 for monitoring a patient using ear-worn devices, according to one embodiment. Although the example logic flow 1100 depicts a particular sequence of operations, the sequence may be altered without departing from the scope of the present disclosure. For example, some of the operations depicted may be performed in parallel or in a different sequence that does not materially affect the function of the logic flow 1100. In other examples, different components of an example device or system that implements the logic flow 1100 may perform functions at substantially the same time or in a specific sequence.

According to some examples, the logic flow 1100 includes monitoring, by one or more sensors of an ear-worn device, data reflecting a plurality of attributes of a patient at block 1102. For example, the sensors 204, accelerometers 216, pulse oximeters 210, and/or electrodes 214 of earbud 114a may monitor data reflecting a plurality of attributes of a patient. The attributes may be any type of attribute, such as physiological attributes, movements, eye blinks, activities, tasks, fatigue levels, etc.

According to some examples, the logic flow 1100 includes analyzing, by a processor of the ear-worn device, the data monitored by the one or more sensors at block 1104. For example, the processor 116e may analyze the data monitored by the one or more sensors. For example, the processor 116e may detect fatigue, slowing reaction times, decreasing counts of eye blinks, longer eye blink durations, brain waves indicative of fatigue (or stress and/or sleep), longer times to complete movements and/or tasks, etc.

According to some examples, the logic flow 1100 includes determining, by the processor based on the analysis, the patient has a first level of fatigue of a plurality of levels of fatigue at block 1106. For example, the processor 116e may determine, based on the analysis, the patient has a first level of fatigue of a plurality of levels of fatigue. For example, the processor 116e may compute a fatigue score based on the data collected at block 1102 and the processing at block 1104. The fatigue score may be used to determine the first fatigue level based ranges of scores associated with different ones of the plurality of fatigue levels. For example, the tracking application 218 may include predetermined score ranges for different fatigue levels, e.g., 0-20 for a first level, 21-40 for a second level, 41-60 for a third level, 61-80 for a fourth level, and 81-100 for a fifth level. Embodiments are not limited in these contexts.

According to some examples, the logic flow 1100 includes generating, by the processor, a recommendation based on the determined first level of fatigue at block 1108. For example, the processor 116e may generate a recommendation based on the determined first level of fatigue. The recommendation may be any type of recommendation, such as waking the person, initiating autonomous driving, etc.

According to some examples, the logic flow 1100 includes outputting, by the processor, an indication of the recommendation at block 1110. For example, the processor 116e may output an indication of the recommendation via speakers 206, haptic feedback modules 212, the network 112 (e.g., to transmit an instruction to a vehicle computing system to engage safety features, transmit a data package to a medical provider, etc.).

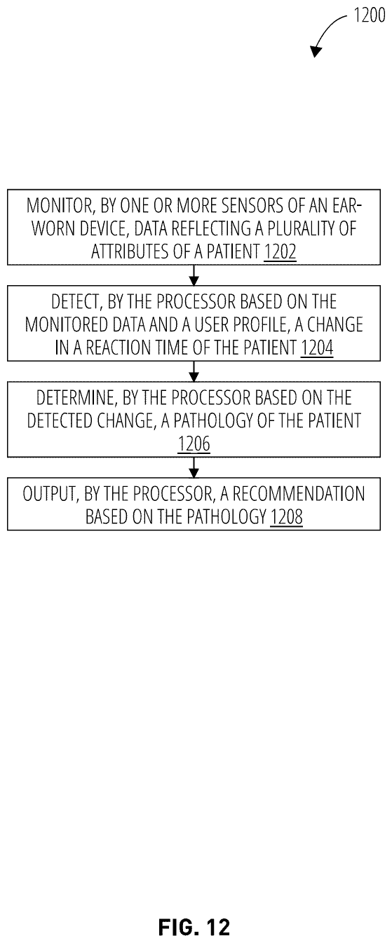

FIG. 12 illustrates an example logic flow 1200 for monitoring a patient using ear-worn devices to detect a pathology, according to one embodiment. Although the example logic flow 1200 depicts a particular sequence of operations, the sequence may be altered without departing from the scope of the present disclosure. For example, some of the operations depicted may be performed in parallel or in a different sequence that does not materially affect the function of the logic flow 1200. In other examples, different components of an example device or system that implements the logic flow 1200 may perform functions at substantially the same time or in a specific sequence.

According to some examples, the logic flow 1200 includes monitoring, by one or more sensors of an ear-worn device, data reflecting a plurality of attributes of a patient at block 1202. For example, the sensors 204, accelerometers 216, pulse oximeters 210, and/or electrodes 214 of earbud 114a may monitor data reflecting a plurality of attributes of a patient. The attributes may be any type of attribute, such as physiological attributes, movements, eye blinks, activities, tasks, fatigue levels, etc.

According to some examples, the logic flow 1200 includes detecting, by the processor based on the monitored data and a user profile, a change in a reaction time of the patient at block 1204. For example, the processor 116e may detect, by the processor based on the monitored data and a user profile 224, a change in a reaction time of the patient. For example, the processor 116e may determine the patient is slower to respond to stimuli, takes longer to complete a movement and/or task, etc.

According to some examples, the logic flow 1200 includes determining, by the processor based on the detected change, a pathology of the patient at block 1206. For example, the processor 116e may determine, based on the change detected at block 1204, a pathology of the patient. For example, the changes detected at block 1204 may be associated with a pathology such as Alzheimer's in the therapies 222. As such, the processor 116e may determine the patient may have Alzheimer's.

According to some examples, the logic flow 1200 includes outputting, by the processor, a recommendation based on the pathology at block 1208. For example, the processor 116e may output, by the processor, a recommendation including a suggestion (e.g., to use an assistive device when walking, contacting a medical provider for diagnosis), transmit an instruction to a device such as RPT device 106 to modify therapy delivered to the patient based on the pathology, provide educational information to the patient, or any other type of recommendation. Embodiments are not limited in these contexts.

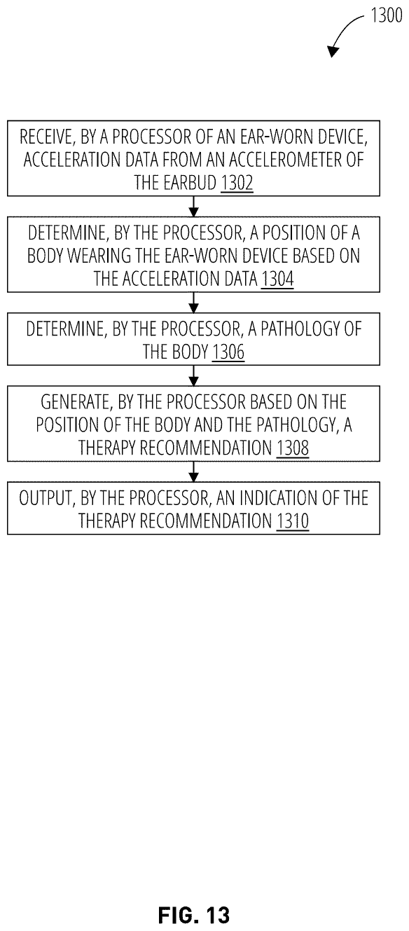

FIG. 13 illustrates an example logic flow 1300 for using ear-worn devices to determine positions and/or movements of a person. Although the example logic flow 1300 depicts a particular sequence of operations, the sequence may be altered without departing from the scope of the present disclosure. For example, some of the operations depicted may be performed in parallel or in a different sequence that does not materially affect the function of the logic flow 1300. In other examples, different components of an example device or system that implements the logic flow 1300 may perform functions at substantially the same time or in a specific sequence. Furthermore, in some embodiments, the logic flow 1300 may be used in combination with other techniques and/or logic flows to determine a sleep position of a patient and/or provide a recommendation.

According to some examples, the logic flow 1300 includes receiving, by a processor of an ear-worn device, acceleration data from an accelerometer of the ear-worn device at block 1302. For example, the processor 116e of earbud 114a may receive acceleration data from accelerometer 216 of the earbud 114a.

According to some examples, the logic flow 1300 includes determining, by the processor, a position of a body wearing the ear-worn device based on the acceleration data at block 1304. For example, the processor 116e may determine position of a body wearing the earbud 114a based on the acceleration data. The position may include positions of different parts of the body.

According to some examples, the logic flow 1300 includes determining, by the processor, a pathology of the body at block 1306. For example, the processor 116e may determine a pathology of the body. The pathology may be determined based on any suitable technique, such as referencing indications of pathologies in the user profile 224, detecting the pathology using soundwave analysis, detecting therapy devices such as RPT device 106, mask 108, etc.

According to some examples, the logic flow 1300 includes generating, by the processor based on the position of the body and the pathology, a recommendation at block 1308. For example, the processor 116e may generate, based on the position of the body and the pathology, a recommendation. For example, the recommendation may be to alert the patient of drowsiness and/or sleep onset, alert the patient of decreasing reaction time, to change sleep positions, to adjust the operating parameters of the RPT device 106, etc.

According to some examples, the logic flow 1300 includes outputting, by the processor, an indication of the recommendation at block 1310. For example, the speakers 206 may output sounds to wake the patient, the haptic feedback modules 212 may generate vibrations to wake the patient, etc. As other examples, the indication of the recommendation may be transmitted to other devices via the communications interface 120e, e.g., to engage safety features in a vehicle, adjust one or more parameters of the RPT device 106, order a new type of mask 108, send the recommendation to a medical provider's computing device, etc.

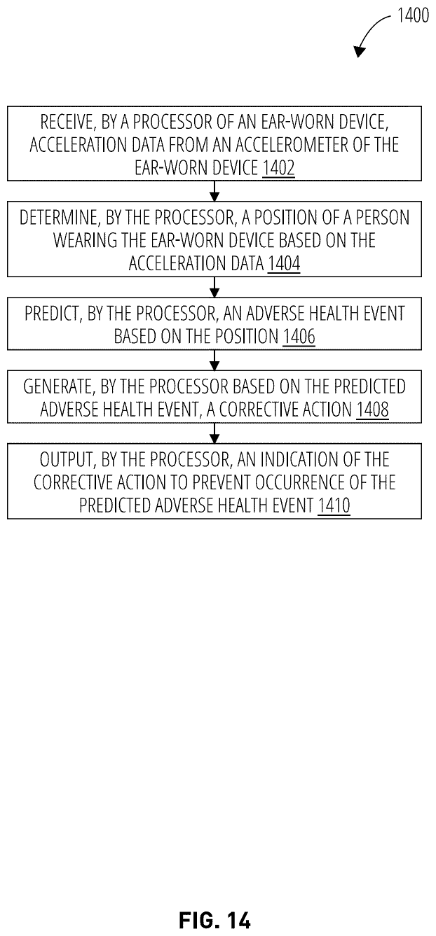

FIG. 14 illustrates an example logic flow 1400 for predicting adverse health events using ear-worn devices, according to one embodiment. Although the example logic flow 1400 depicts a particular sequence of operations, the sequence may be altered without departing from the scope of the present disclosure. For example, some of the operations depicted may be performed in parallel or in a different sequence that does not materially affect the function of the logic flow 1400. In other examples, different components of an example device or system that implements the logic flow 1400 may perform functions at substantially the same time or in a specific sequence.

According to some examples, the logic flow 1400 includes receiving, by a processor of an ear-worn device, acceleration data from an accelerometer of the ear-worn device at block 1402. For example, the processor 116e of earbud 114a may receive acceleration data from an accelerometer 216 of the earbud 114a.

According to some examples, the logic flow 1400 includes determining, by the processor, a position of a body wearing the ear-worn device based on the acceleration data at block 1404. For example, the processor 116e may determine a position of a person wearing the earbud 114a based on the acceleration data. For example, based on the acceleration data, the processor 116e may determine the person is waking from sleep.

According to some examples, the logic flow 1400 includes predicting, by the processor, an adverse health event based on the position of the body at block 1406. For example, the processor 116e may predict an adverse health event based on the position of the body. For example, the processor 116e may predict that the person, who is waking up from sleep, may fall while getting out of bed. In some embodiments, the processor 116e may reference the user profile 224 of the user to determine the number of times the person falls out of bed exceeds a threshold.

According to some examples, the logic flow 1400 includes generating, by the processor based on the predicted adverse health event, a corrective action at block 1408. For example, the processor 116e may generate, based on the predicted adverse health event, a corrective action to prevent the occurrence of the adverse health event. Example corrective actions include notifying the person, emitting an alarm, etc.

According to some examples, the logic flow 1400 includes outputting, by the processor, an indication of the corrective action to prevent occurrence of the predicted adverse health event at block 1410. For example, the processor 116e may output, an indication of the corrective action to prevent occurrence of the predicted adverse health event. Example indications include outputting sounds via the speakers 206, emitting vibrations via the haptic feedback modules 212, etc. Embodiments are not limited in these contexts.

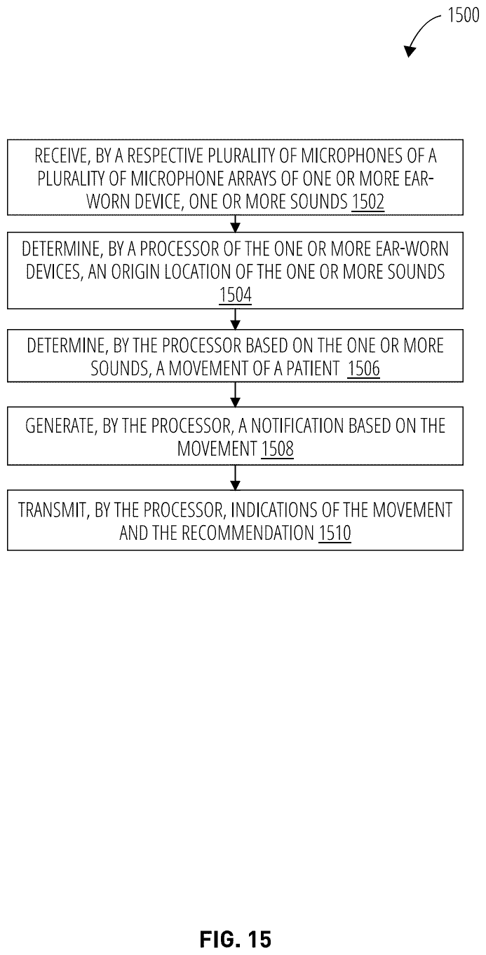

FIG. 15 illustrates an embodiment of a logic flow 1500, according to one embodiment. The logic flow 1500 represents some or all of the operations executed by one or more embodiments described herein. For example, the logic flow 1500 includes some or all of the operations performed by devices or entities in the system 100 to use ear-worn devices determine a movement of a patient based at least in part on soundwave detection. Embodiments are not limited in these contexts. Furthermore, in some embodiments, the logic flow 1500 may be used in combination with other techniques and/or logic flows to determine a position of a patient and/or provide a recommendation.

In block 1502, logic flow 1500 receives, by a respective plurality of microphones of a plurality of microphone arrays 208 of one or more ear-worn devices such as earbuds 114a, 114b, one or more sounds. The sounds may include ambient sounds, sounds generated by devices in the system 100, sounds generated by the body of the wearer of the earbuds, etc. In block 1504, logic flow 1500 determines, by a processor 116e of the one or more earbuds 114a, 114b, a location the one or more sounds originated from. For example, the tracking application 218 and/or the models 220 may process one or more of the sounds and determine the location based on triangulation, trilateration, beamforming, single or multiple microphone acoustic impedance measurements, impulse/frequency response function measurements, or any suitable technique.

In block 1506, logic flow 1500 determines, by the processor based on the one or more sounds, a movement of a patient. For example, the tracking application 218 and/or the models 220 may process one or more of the sounds and determine the movement based on the sounds, e.g., by identifying similar sounds in the user profile 224 associated a with particular sleep position. For example, the sound may be a falling sound and may match a sound in the user profile 224 that is associated with a person falling. In block 1508, logic flow 1500 generates, by the processor based on the sounds and movement, a notification based on the movement determined at block 1506. For example, the tracking application 218 may determine to alert a trusted contact, medical provider, etc., to call assistance to the person that fell.

At block 1510, the processor transmits an indication of the notification to a device via a network. For example, the tracking application 218 may call an ambulance to the home of the person who fell. More generally, the notification may be outputted via the earbuds 114a-114b, external devices 104 of the user, other wearables 110 of the user, etc. Embodiments are not limited in these contexts.

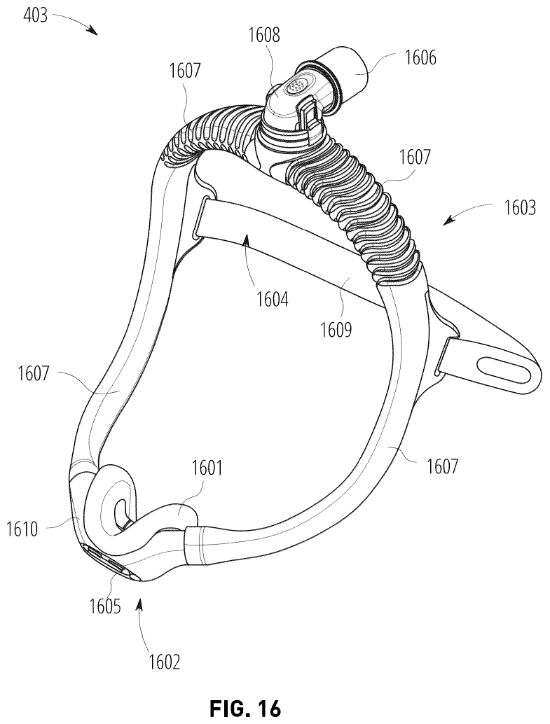

FIG. 16 shows a patient interface 403 having conduit headgear 1604, in accordance with one embodiment. The patient interface 403 is one example of the mask 108 of FIG. 1, and therefore includes a processor 116d, memory 118d, and communications interface 120d (not pictured for clarity).

As shown, a non-invasive patient interface 403 includes a seal-forming structure 1601, a plenum chamber 1602, a positioning and stabilizing structure 1603, a vent 1605, an elbow 1608, a strap 1609, a cushion module 1610, and one embodiment of connection port 1606 for connection to air circuit 404. In some forms a functional aspect may be provided by one or more physical components. In some forms, one physical component may provide one or more functional aspects. In use the seal-forming structure 1601 is arranged to surround an entrance to the airways of the patient so as to maintain positive pressure at the entrance(s) to the airways of the patient 401. The sealed patient interface 403 is therefore suitable for delivery of positive pressure therapy, e.g., in the form of supplementary gas 1737 (e.g., oxygen).

As stated, the patient interface 403 can communicate with other devices, such as the earbuds 114a-114b and the RPT device 106, e.g., to receive instructions and modify respiratory therapy provided via the patient interface based on the instructions. The patient interface 403 is constructed and arranged to be able to provide a supply of air at a positive pressure above the ambient, for example at least 2, 4, 6, 10, or 20 cmH2O with respect to ambient.

In some embodiments, the positioning and stabilizing structure 1603 comprise one or more headgear tubes 1607 that deliver pressurized air received from a conduit forming part of the air circuit 404 from the RPT device to the patient's airways, for example through the plenum chamber 1602 and seal-forming structure 1601. In the embodiment illustrated in FIG. 16, the positioning and stabilizing structure 1603 comprises two tubes 1607 that deliver air to the plenum chamber 1602 from the air circuit 404. The tubes 1607 are configured to position and stabilize the seal-forming structure 1601 of the patient interface 403 at the appropriate part of the patient's face (for example, the nose and/or mouth) in use. This allows the conduit of air circuit 404 providing the flow of pressurized air to connect to a connection port 1606 of the patient interface in a position other than in front of the patient's face, for example on top of the patient's head.

As shown, the patient interface 403 includes a vent 1605 constructed and arranged to allow for the washout of exhaled gases, e.g., carbon dioxide. In some embodiments, the vent 1605 is configured to allow a continuous vent flow from an interior of the plenum chamber 1602 to ambient whilst the pressure within the plenum chamber is positive with respect to ambient. The vent 1605 is configured such that the vent flow rate has a magnitude sufficient to reduce rebreathing of exhaled CO2 by the patient while maintaining the therapeutic pressure in the plenum chamber in use.

Connection port 1606 allows for connection to the air circuit 404. In one or more embodiments, the patient interface 403 includes a forehead support. In one or more embodiments, the patient interface 403 includes an anti-asphyxia valve. Air may be delivered to the patient in one of two main ways. In one example, the patient may receive the flow of pressurized air through headgear tubes 1607. This may be referred to as a “tube up” configuration and may position a connection port at the top of the patient's head. In another example, the patient may receive the flow of pressurized air through a conduit connected to the plenum chamber 1602, for example through the connection port 1606. This may be referred to a “tube down” configuration where the airflow conduit is positioned in front of the patient's face.

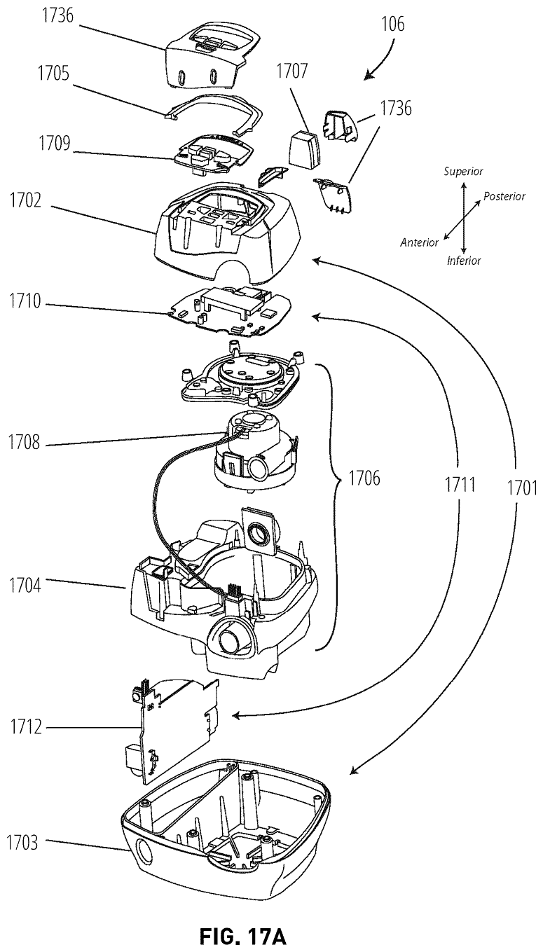

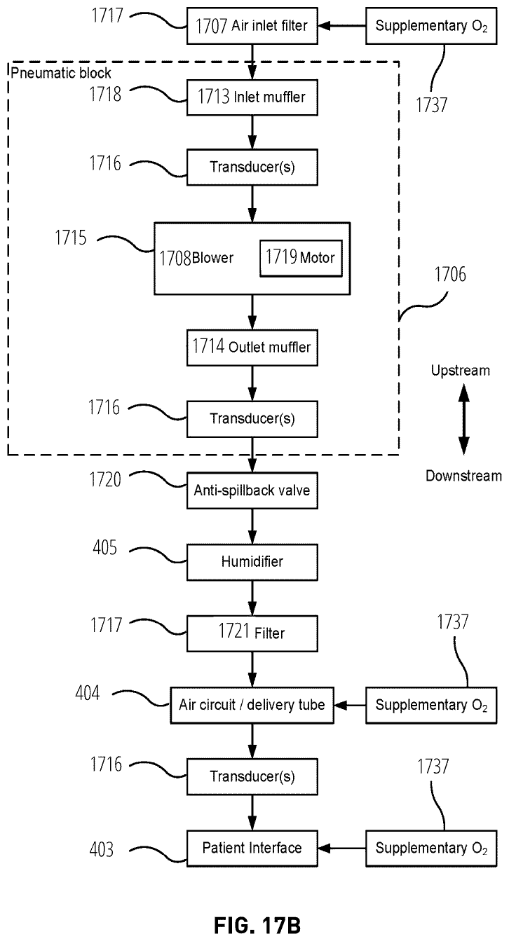

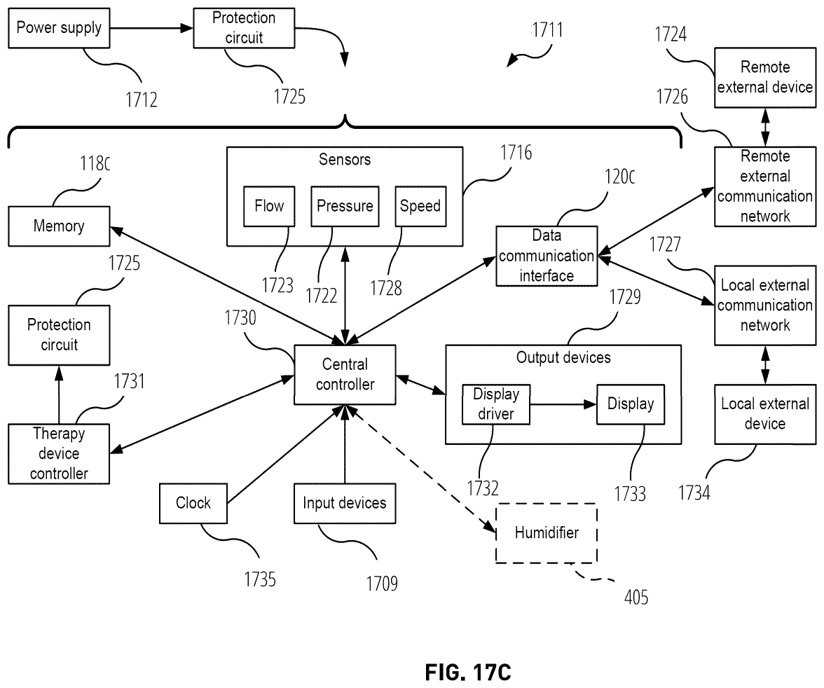

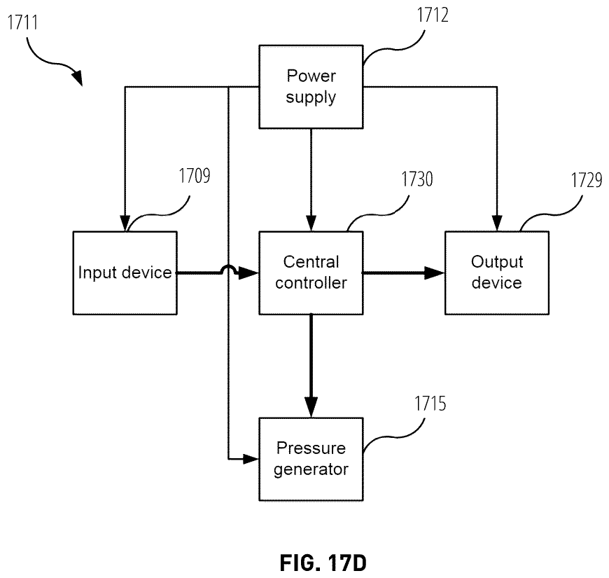

FIG. 17A shows an RPT device 106 in accordance with one embodiment. The RPT device 106 comprises mechanical, pneumatic, and/or electrical components and is configured to execute one or more algorithms, such as any of the methods, in whole or in part, described herein. The RPT device 106 may be configured to generate a flow of air for delivery to a patient's airways, such as to treat one or more of the respiratory conditions described elsewhere herein. For example, the tracking application 218 may cause the RPT device 106 to generate the flow of air to treat a pathology detected according to the techniques disclosed herein. Doing so may include the RPT device 106 modifying the delivery of therapy using one or more of the components depicted in FIG. 17A-FIG. 17D, which may include modifying any attribute thereof.

As shown in FIG. 17A, the RPT device 106 may have an external housing 1701, formed in two parts, an upper portion 1702 and a lower portion 1703. Furthermore, the external housing 1701 may include one or more panel(s) 1736. The RPT device 106 comprises a chassis 1704 that supports one or more internal components of the RPT device 106. The RPT device 106 may include a handle 1705. One or more of the air path items may be located within a removable unitary structure which will be referred to as a pneumatic block 1706. The pneumatic block 1706 may be located within the external housing 1701. In one embodiment a pneumatic block 1706 is supported by, or formed as part of the chassis 1704.

FIG. 17B is a schematic diagram of the pneumatic path of an RPT device 106 in accordance with one or more embodiments. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

As shown in FIG. 17B, the pneumatic path of the RPT device 106 may comprise one or more air path items, e.g., an inlet air filter 1707, an inlet muffler 1713, a pressure generator 1715 capable of supplying air at positive pressure (e.g., a blower 1708), an outlet muffler 1714 and one or more transducers 1716, such as pressure sensors 1722 and flow rate sensors 1723.

The RPT device 106 may include an air filter 1717, or a plurality of air filters 1717. In the embodiment illustrated in FIG. 17B, an inlet air filter 1707 is located at the beginning of the pneumatic path upstream of a pressure generator 1715. In some embodiments, an outlet air filter 1721, for example an antibacterial filter, is located between an outlet of the pneumatic block 1706 and a patient interface 403. The RPT device 106 may include a muffler 1718, or a plurality of mufflers 1718. In one or more embodiments, an inlet muffler 1713 is located in the pneumatic path upstream of a pressure generator 1715. In one or more embodiments, an outlet muffler 1714 is located in the pneumatic path between the pressure generator 1715 and a patient interface 403.

In some embodiments, a pressure generator 1715 for producing a flow, or a supply, of air at positive pressure is a controllable blower 1708. For example, the blower 1708 may include a brushless DC motor 1719 with one or more impellers. The impellers may be located in a volute. The blower may be capable of delivering a supply of air, for example at a rate of up to about 120 liters/minute, at a positive pressure in a range from about 4 cmH2O to about 20 cmH2O, or in other forms up to about 30 cmH2O when delivering respiratory pressure therapy.

The pressure generator 1715 may be under the control of the therapy device controller 1731. In other forms, a pressure generator 1715 may be a piston-driven pump, a pressure regulator connected to a high pressure source (e.g., compressed air reservoir), or a bellows. The therapy device controller 1731 may receive instructions from the tracking application 218 and adjust the therapy based on the instruction.

In some embodiments, one or more transducers 1716 are located upstream and/or downstream of the pressure generator 1715. The one or more transducers 1716 may be constructed and arranged to generate signals representing properties of the flow of air such as a flow rate, a pressure or a temperature at that point in the pneumatic path. In some embodiments, one or more transducers 1716 may be located proximate to the patient interface 403. In one or more embodiments, a signal from a transducer 1716 may be filtered, such as by low-pass, high-pass or band-pass filtering. In some embodiments, a motor speed transducer 1728 is used to determine a rotational velocity of the motor 1719 and/or the blower 1708. A motor speed signal from the motor speed transducer 1728 may be provided to the therapy device controller 1731. The motor speed transducer 1728 may, for example, be a speed sensor, such as a Hall effect sensor.

As shown in FIG. 17B an anti-spill back valve 1720 is located between the humidifier 405 and the pneumatic block 1706. The anti-spill back valve is constructed and arranged to reduce the risk that water will flow upstream from the humidifier 405, for example to the motor 1719.

FIG. 17C is a schematic diagram of the electrical components 1711 of an RPT device such as RPT device 106 in accordance with one embodiment.

As shown in FIG. 17C, the RPT device 106 comprises an electrical power supply 1712, one or more input devices 1709, a central controller 1730, a therapy device controller 1731, a pressure generator 1715, one or more protection circuits 1725, memory 118c, transducers 1716, communications interface 120c and one or more output devices 1729. Electrical components 1711 may be mounted on a single Printed Circuit Board Assembly (PCBA) 1710. In an alternative form, the RPT device 106 may include more than one PCBA 1710.

The power supply 1712 may be located internal or external of the external housing 1701 of the RPT device 106. In one or more embodiments, power supply 1712 provides electrical power to the RPT device 106 only. In another embodiment, power supply 1712 provides electrical power to both RPT device 106 and humidifier 405.

In some embodiments, one or more flow rate sensors 1723 may be based on a differential pressure transducer. In one or more embodiments, a signal generated by the flow rate sensor 1723 and representing a flow rate is received by the central controller 1730. The RPT device 106 may include a clock 1735 that is connected to the central controller 1730.

In some embodiments, therapy device controller 1731 is a therapy control module that forms part of one or more algorithms executed by the central controller 1730. In one or more embodiments, therapy device controller 1731 is a dedicated motor control integrated circuit. The therapy device controller 1731 and the central controller 1730 represent the processor 116b of FIG. 1.

The one or more protection circuits 1725 may comprise an electrical protection circuit, a temperature and/or pressure safety circuit. Memory 118c may be located on the PCBA 1710. Memory 118c may be in any form. Additionally, or alternatively, RPT device 106 includes a removable form of memory 118c, for example a memory card made in accordance with the Secure Digital (SD) standard.

In one or more embodiments, the communications interface 120c is connected to the central controller 1730. Communications interface 120c may be connectable to a remote external communication network 1726 and/or a local external communication network 1727 (e.g., the network 112). The remote external communication network 1726 may be connectable to a remote external device 1724. The local external communication network 1727 may be connectable to a local external device 1734.

In one or more embodiments, remote external communication network 1726 is the Internet. The communications interface 120c may use wired communication or wireless communications to connect to the Internet. In one or more embodiments, local external communication network 1727 utilizes one or more communication standards, such as Bluetooth, a consumer infrared protocol, an I/O bus such as a universal serial bus (USB), peripheral component interconnects (PCIs), etc.

In one or more embodiments, remote external device 1724 is one or more computers, for example a cluster of networked computers. In one or more embodiments, remote external device 1724 may be virtual computers, rather than physical computers. In either case, such a remote external device 1724 may be accessible to an appropriately authorized person such as a clinician.

The local external device 1734 represents the external devices 104, which may be a personal computer, mobile phone, tablet, or remote control.

An output device 1729 may take the form of one or more of a visual, audio, and haptic unit. A visual display 1733 may be a Liquid Crystal Display (LCD) or Light Emitting Diode (LED) display. A display driver 1732 receives as an input the characters, symbols, or images intended for display on the display 1733, and converts them to commands that cause the display 1733 to display those characters, symbols, or images.

A display 1733 is configured to visually display characters, symbols, or images in response to commands received from the display driver 1732. For example, the display 1733 may be an eight-segment display, in which case the display driver 1732 converts each character or symbol, such as the figure “0”, to eight logical signals indicating whether the eight respective segments are to be activated to display a particular character or symbol.

In some embodiments, the air circuit 404 is a conduit or a tube constructed and arranged to allow, in use, a flow of air to travel between two components such as RPT device 106 and the patient interface 403. In particular, the air circuit 404 may be in fluid connection with the outlet of the pneumatic block 1706 and the patient interface. The air circuit may be referred to as an air delivery tube. In some cases there may be separate limbs of the circuit for inhalation and exhalation. In other cases a single limb is used.

In some embodiments, the air circuit 404 may comprise one or more heating elements configured to heat air in the air circuit, for example to maintain or raise the temperature of the air. The heating element may be in a form of a heated wire circuit, and may comprise one or more transducers, such as temperature sensors. In one or more embodiments, the heated wire circuit may be helically wound around the axis of the air circuit 404. The heating element may be in communication with a controller such as a central controller 1730.

As illustrated in FIG. 17D, the power supply 1712 may provide electrical power to the input devices 1709, the central controller 1730, the output device 1729, and the pressure generator 1715. The power supply 1712 may also provide electric energy to other components of the RPT device 106 (or the humidifier 405).

In one or more embodiments, an RPT device 106 includes one or more input devices 1709 in the form of buttons, switches or dials to allow a person to interact with the device. The buttons, switches or dials may be physical devices, or software devices accessible via a touch screen. The buttons, switches or dials may, in one or more embodiments, be physically connected to the external housing 1701, or may, in another form, be in wireless communication with a receiver that is in electrical connection to the central controller 1730.

In one or more embodiments, the input device 1709 may be constructed and arranged to allow a person to select a value and/or a menu option.

In one or more embodiments, the central controller 1730 is one or a plurality of processors suitable to control an RPT device 106. The central controller 1730 is shown in FIG. 17C and FIG. 17D. The central controller 1730 may be configured to receive input signal(s) from one or more transducers 1716, one or more input devices 1709, and/or the humidifier 405. The central controller 1730 may be configured to provide output signal(s) to one or more of an output device 1729, a pressure generator 1715, a therapy device controller 1731, a communications interface 120c, and/or the humidifier 405. Furthermore, central controller 1730 can receive information from or transmit information to earbuds 114a-114b.

In some embodiments, the central controller 1730 is configured to implement the one or more methodologies described herein, such as one or more algorithms which may be implemented with processor-control instructions, expressed as computer programs stored in a non-transitory computer readable storage medium, such as memory 118c. In some embodiments, the central controller 1730 may be integrated with an RPT device 106. However, in some embodiments, some methodologies may be performed by a remotely located device. For example, the remotely located device may determine control settings for a ventilator or detect respiratory related events by analysis of stored data such as from any of the sensors described herein.

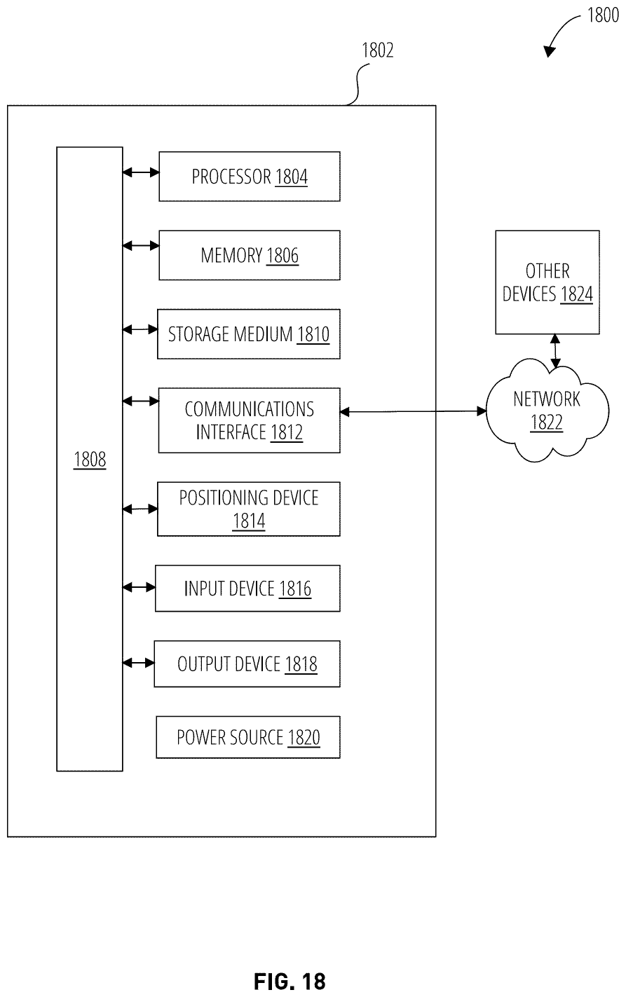

FIG. 18 illustrates an example computing system 1800 suitable for implementing various embodiments as described herein. As shown, the computing system 1800 comprises a computer 1802, which is representative of any type of physical and/or virtualized computing device. Examples of the computer 1802 include, but are not limited to, a server, workstation, laptop, mobile device, smartphone, tablet computer, mainframe, distributed computing system, compute cluster, media device, camera, gaming device, a portable digital assistant (PDA), a system-on-chip (SoC), a pager, a television, a wearable device, a virtual machine (VM), container, or any other device with processing capabilities. In one embodiment, the computer 1802 is representative of some or all of the components of the system 100. More generally, the computing system 1800 is configured to implement all systems, methods, apparatuses, media, and embodiments disclosed herein.

For example, computer 1802 may represent some or all of the components of the earbuds 114a-114b, external devices 104, RPT device 106, mask 108, other wearables 110, smartwatch 304, smartwatch 412, chest monitor 410, and/or device 504. However, all components of the computer 1802 depicted in FIG. 18 need not be included in the earbuds 114a-114b, external devices 104, RPT device 106, mask 108, other wearables 110, and/or device 504. Embodiments are not limited in these contexts.

As shown, the computer 1802 includes one or more processors 1804, one or more memories 1806, one or more non-transitory storage media 1810, one or more communications interfaces 1812, one or more positioning devices 1814, one or more input devices 1816, and one or more output devices 1818 communicably coupled via an interconnect 1808. A power source 1820, such as a power supply, battery, or any type of power source may provide power to the computer 1802.

The processor 1804 is representative of any type of processing circuit. For example, the processor 1804 may be a central processing unit (CPU), a microprocessor, a graphics processing unit (GPU), a microcontroller, an application-specific integrated circuit (ASIC), a programmable logic device (PLD), a digital signal processor (DSP), a field programmable gate array (FPGA), a state machine, a controller, gated or transistor logic, a digital signal processor, analog to digital converter, digital to analog converter, and the like.

The memory 1806 is representative of any computer readable medium to store data, code, or other information. The memory 1806 may include volatile memory, such as volatile Random Access Memory (RAM) including a cache area for the temporary storage of data. The memory 1806 may also include non-volatile memory, which can be embedded and/or may be removable. The non-volatile memory can additionally or alternatively include an electrically erasable programmable read-only memory (EEPROM), flash memory or the like. The storage medium 1810 is representative of any type of computer readable medium to store data, code, or other information. Examples of storage media 1810 include solid state drives, hard drives, Redundant Array of Independent Disks (RAID) drives, memory pools, USB storage devices, and the like.

The memory 1806 and storage medium 1810 can store any number and type of computer-executable instructions executed by the processor 1804 to implement the functions of the computer 1802 described herein. For example, the memory 1806 and/or storage medium 1810 may include the tracking application 218, the model 220, the therapies 222, the user profiles 224, and/or the device profiles 226.

The interconnect 1808 is representative of any type of circuitry to connect the components of the computer 1802. For example, the interconnect 1808 can include or represent, a system bus, a universal serial bus (USB) interface, a peripheral component interconnect (PCI), a Peripheral Component Interconnect-enhanced (PCIe), etc. As discussed herein, the interconnect 1808 may operatively couple various components with one another, or in other words, electrically connects those components, either directly or indirectly—by way of intermediate component(s)—with one another.

The one or more input devices 1816 are representative of any type of input device for receiving input, such as a keypad, keyboard, touchscreen, touchpad, microphone, camera, fingerprint sensor, mouse, joystick, other pointer device, button, soft key, and the like. The one or more output devices 1818 are representative of any type of device for outputting information, such as a monitor, speaker, haptic feedback module, printer, and the like.

The computer 1802 may use the communications interface 1812 to communicate with one or more other devices 1824 via a network 1822. The communications interface 1812 allows the computer 1802 to communicate with and conduct transactions with other devices and systems, such as the other devices 1824. The communications interface 1812 may be a wired and/or a wireless interface. Thus, communications can be conducted, for example, via the wireless communications interface 1812, which can be or include a radio-frequency transceiver, a Bluetooth device, Wi-Fi device, a Near-Field Communication (NFC) device, and other wireless transceivers. In addition, a positioning device 1814 such as a Global Positioning System (GPS) device may be included for navigation and location-related data exchanges, ingoing and/or outgoing. Wi-Fi networks use radio technologies such as IEEE 802.11x (a, b, g, n, ac, ax, etc.) to provide secure, reliable, fast wireless connectivity. A Wi-Fi network connects computers to each other, to the Internet, and to wired networks (which use IEEE 802.3-related media and functions). Communications may also and/or alternatively be conducted via wired connections using the communications interface 1812, e.g., using USB, Ethernet, and other physically connected modes of data transfer. The network 1822 may be any one of, or the combination of, wired and/or wireless networks including without limitation a direct connection, a private network (e.g., an intranet), a public network (e.g., the Internet), a Personal Area Network (PAN), a Local Area Network (LAN), a Wide Area Network (WAN), a wireless network, a cellular network, and other communications networks.

The computer 1802 is configured to use the communications interface 1812 as, for example, a network interface to communicate with one or more other devices on a network such as network 1822. In this regard, the computer 1802 utilizes the wireless communications interface 1812 as an antenna operatively coupled to a transmitter and a receiver (together a “transceiver”) included with the communications interface 1812. The communications interface 1812 is configured to provide signals to and receive signals from the transmitter and receiver, respectively. The signals may include signaling information in accordance with the air interface standard of the applicable cellular system of a wireless network. In this regard, the computer 1802 may be configured to operate with one or more air interface standards, communication protocols, modulation types, and access types. By way of illustration, the computer 1802 may be configured to operate in accordance with any of a number of first, second, third, fourth, fifth-generation communication protocols and/or the like. For example, as a smartphone, the computer 1802 may be configured to operate in accordance with fourth-generation (4G) wireless communication protocols such as Long-Term Evolution (LTE), fifth-generation (5G) wireless communication protocols, Bluetooth Low Energy (BLE) communication protocols such as Bluetooth 5.0, ultra-wideband (UWB) communication protocols, and/or the like. The computer 1802 may also be configured to operate in accordance with non-cellular communication mechanisms, such as via a wireless local area network (WLAN) or other communication/data networks.

The computer 1802 may be under the control of any suitable operating system (not pictured). Example operating systems include, but are not limited to, Linux® operating systems, UNIX®, Windows® operating systems, macOS®, iOS®, Android®, and any other type of operating system.

The computer 1802 as illustrated diagrammatically represents at least one example of a possible implementation, where alternatives, additions, and modifications are possible for performing some or all of the described methods, operations, and functions. Although shown separately, in some embodiments, two or more computers 1802, systems, servers, or illustrated components may be utilized. In some implementations, the functions of one or more systems, servers, or illustrated components may be provided by a single system or server. In some embodiments, the functions of one illustrated system or server may be provided by multiple systems, servers, or computing devices, including those physically located at a central facility, those logically local, and those located as remote with respect to each other.

Aspects of the present disclosure are described herein with reference to flowchart illustrations and/or block diagrams of computer-implemented methods and computing systems according to embodiments of the disclosure. It will be understood that each block of the flowchart illustrations and/or block diagrams, and combinations of blocks in the flowchart illustrations and/or block diagrams, can be implemented by computer readable program instructions that may be provided to a processor of a computer or other programmable data processing apparatus (the term “apparatus” includes systems and computer program products). The processor may execute the computer readable program instructions thereby creating a means for implementing the actions specified in the flowchart illustrations and/or block diagrams. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the actions specified in the flowchart illustrations and/or block diagrams. In particular, the computer readable program instructions may be used to produce a computer-implemented method by executing the instructions to implement the actions specified in the flowchart illustrations and/or block diagrams.

The computer program instructions may also be stored in a computer-readable memory that can direct a computer or other programmable data processing apparatus to function in a particular manner, such that the instructions stored in the computer readable memory produce an article of manufacture including instructions, which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions, which execute on the computer or other programmable apparatus, provide steps for implementing the functions/acts specified in the flowchart and/or block diagram block or blocks. Alternatively, computer program implemented steps or acts may be combined with operator or human implemented steps or acts to carry out an embodiment.

In the flowchart illustrations and/or block diagrams disclosed herein, each block in the flowchart/diagrams may represent a module, segment, or portion of instructions, which comprises one or more executable instructions for implementing the specified logical function(s). In some implementations, the functions noted in the blocks may occur out of the order noted in the Figures. For example, two blocks shown in succession may, in fact, be executed substantially concurrently, or the blocks may sometimes be executed in the reverse order, depending upon the functionality involved.

Computer program instructions are configured to carry out operations of the present disclosure and may be or may incorporate assembler instructions, instruction-set-architecture (ISA) instructions, machine instructions, machine dependent instructions, microcode, firmware instructions, state-setting data, source code, and/or object code written in any combination of one or more programming languages.

An application program may be deployed by providing computer infrastructure operable to perform one or more embodiments disclosed herein by integrating computer readable code into a computing system thereby performing the computer-implemented methods disclosed herein.

Although various computing environments are described above, these are only examples that can be used to incorporate and use one or more embodiments. Many variations are possible.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described to explain the principles of one or more aspects of the disclosure and the practical application, and to enable others of ordinary skill in the art to understand one or more aspects of the disclosure for various embodiments with various modifications as are suited to the particular use contemplated.

What is claimed is:

1. A system, comprising:
a first ear-worn device; and
a second ear-worn device,
the first ear-worn device comprising:
an accelerometer to provide acceleration data; and
a processor operable to execute one or more instructions to cause the processor to:
determine, based on the acceleration data, a duration of a movement of a body wearing the first ear-worn device;
determine, based on the duration of the movement and a profile, a change in the duration of the movement;
generate, based on the change in the duration of the movement, a recommendation; and
output an indication of the recommendation.

2. The system of claim 1, wherein the profile comprises durations of a plurality of prior movements associated with the body, wherein the determined change is based on a comparison of the determined duration of the movement and one or more of the durations of the prior movements, wherein the indication of the recommendation is outputted on one or more of: (i) the first and second ear-worn devices, (ii) a smartphone, or (iii) a wearable device.

3. The system of claim 1, the processor operable to execute the one or more instructions to cause the processor to:
receive position data from one or more other devices; and
determine, based on the position data from the one or more other devices, one or more types of movements, wherein the recommendation is further generated by the processor based on the determined one or more types of movements.

4. The system of claim 3, wherein the duration of the movement is based at least in part on a head of the body, wherein the one or more other devices comprise a smartwatch, wherein the one or more types of movements are determined based at least in part on movements of a wrist of the body.

5. The system of claim 4, the processor operable to execute the one or more instructions to cause the processor to:
predict, based on the duration of the movement of the body and the movements of the wrist, a fall event, wherein the recommendation is further based on the predicted fall event.

6. The system of claim 3, the processor operable to execute the one or more instructions to cause the processor to:
receive, from an electroencephalography (EEG) sensor of the first ear-worn device, one or more EEG signals detected by the EEG sensor, wherein the recommendation is further based on the EEG signals.

7. The system of claim 6, the processor operable to execute the one or more instructions to cause the processor to:
determine a first level of fatigue of a plurality of levels of fatigue based on the EEG signals, wherein the recommendation is further based on the determined first level of fatigue.

8. A non-transitory computer-readable storage medium, the computer-readable storage medium including instructions that when executed by a processor of an ear-worn device, cause the processor to:
receive acceleration data from an accelerometer of the ear-worn device;
determine, based on the acceleration data, a duration of a movement of a body wearing the ear-worn device;
determine, based on the duration of the movement and a profile, a change in the duration of the movement;
generate, based on the change in the duration of the movement, a recommendation; and
output an indication of the recommendation.

9. The non-transitory computer-readable storage medium of claim 8, wherein the profile comprises durations of a plurality of prior movements associated with the body, wherein the determined change is based on a comparison of the determined duration of the movement and one or more of the durations of the prior movements, wherein the indication of the recommendation is outputted on one or more of: (i) the ear-worn device, (ii) a smartphone, or (iii) a wearable device.

10. The non-transitory computer-readable storage medium of claim 8, wherein the instructions further cause the processor to:
receive position data from one or more other devices; and
determine, based on the position data from the one or more other devices, one or more types of movements, wherein the recommendation is further generated by the processor based on the determined one or more types of movements.

11. The non-transitory computer-readable storage medium of claim 10, wherein the duration of the movement is based at least in part on a head of the body, wherein the one or more other devices comprise a smartwatch, wherein the one or more types of movements are determined based at least in part on movements of a wrist of the body.

12. The non-transitory computer-readable storage medium of claim 11, wherein the instructions further cause the processor to:
predict, based on the duration of the movement of the body and the movements of the wrist, a fall event, wherein the recommendation is further based on the predicted fall event.

13. The non-transitory computer-readable storage medium of claim 10, wherein the instructions further cause the processor to:
receive, from an electroencephalography (EEG) sensor of the ear-worn device, one or more EEG signals detected by the EEG sensor, wherein the recommendation is further based on the EEG signals.

14. The non-transitory computer-readable storage medium of claim 13, wherein the instructions further cause the processor to:
determine a first level of fatigue of a plurality of levels of fatigue based on the EEG signals, wherein the recommendation is further based on the determined first level of fatigue.

15. A method, comprising:
receiving, by a processor of an ear-worn device, acceleration data from an accelerometer of the ear-worn device;
determining, by the processor, a duration of a movement of a body wearing the ear-worn device;
determining, by the processor based on the duration of the movement and a profile, a change in the duration of the movement;
generating, by the processor based on the change in the duration of the movement, a recommendation; and
outputting, by the processor, an indication of the recommendation.

16. The method of claim 15, wherein the profile comprises durations of a plurality of prior movements associated with the body, wherein the determined change is based on a comparison of the determined duration of the movement and one or more of the durations of the prior movements, wherein the indication of the recommendation is outputted on one or more of: (i) the ear-worn device, (ii) a smartphone, or (iii) a wearable device.

17. The method of claim 15, further comprising:
receiving, by the processor, position data from one or more other devices; and
determining, by the processor based on the position data from the one or more other devices, one or more types of movements, wherein the recommendation is further generated by the processor based on the determined one or more types of movements.

18. The method of claim 17, wherein the duration of the movement is based at least in part on a head of the body, wherein the one or more other devices comprise a smartwatch, wherein the one or more types of movements are determined based at least in part on movements of a wrist of the body.

19. The method of claim 18, further comprising:
predicting, by the processor based on the duration of the movement of the body and the movements of the wrist, a fall event, wherein the recommendation is further based on the predicted fall event.

20. The method of claim 17, further comprising:
receiving, by the processor from an electroencephalography (EEG) sensor of the ear-worn device, one or more EEG signals detected by the EEG sensor, wherein the recommendation is further based on the EEG signals.
